(19) United States
(12) Patent Application Publication
(10) Pub. No.: US 2025/0134475 A1
(43) Pub. Date: May 1, 2025

(54) ANALYTE MONITORING SYSTEMS

(71) Applicant: ABBOTT DIABETES CARE INC., Alameda, CA (US)

(72) Inventors: William Koo Lee, Alameda, CA (US); Saranpreet S. Nagra, Alameda, CA (US); Swati Satish, Fremont, CA (US); Donald R. Curtis, Berkeley, CA (US); Taylor M. Charlesworth, Oakland, CA (US); Vaishnavi Ramesh, San Jose, CA (US); Sabine Kabel-Eckes, Mountain View, CA (US); Genaro S. Sepulveda, Berkeley, CA (US); Chano Y. Puente de la Vega, San Francisco, CA (US)

(21) Appl. No.: 18/676,061

(22) Filed: May 28, 2024

Related U.S. Application Data
(60) Provisional application No. 63/541,087, filed on Sep. 28, 2023.
(60) Provisional application No. 63/469,779, filed on May 30, 2023.

(51) Int. Cl. A61B 5/00; A61B 5/1171; A61B 5/145

(52) U.S. Cl. CPC A61B 5/746; A61B 5/0004; A61B 5/1176; A61B 5/14503; A61B 5/7435; A61B 5/7475

(57) ABSTRACT

Improved digital interfaces, graphical user interfaces, and alarms for analyte monitoring systems are provided. For example, disclosed herein are various embodiments of methods, systems, and interfaces for Silent Mode for alarms, Temporary Mode for alarms, escalating alarms, and alarm snooze features for an analyte monitoring software application. Also, various embodiments of interface enhancements are described, including caregiver alarms, among other embodiments.

### CROSS-REFERENCE TO RELATED APPLICATIONS

The application claims priority to U.S. Application Ser. No. 63/541,087, filed Sep. 28, 2023, and U.S. Application Ser. No. 63/469,779, filed May 30, 2023, which are hereby expressly incorporated by reference in their entirety for all purposes.

### FIELD

The subject matter described herein relates generally to analyte monitoring systems, as well as systems, methods, and devices relating thereto, including digital interfaces, user interfaces, and alarms for such systems.

### BACKGROUND

The detection and/or monitoring of analyte levels, such as glucose, ketones, lactate, oxygen, hemoglobin A1C, or the like, can be vitally important to the overall health of a person, particularly for an individual having diabetes. Patients suffering from diabetes mellitus can experience complications including loss of consciousness, cardiovascular disease, retinopathy, neuropathy, and nephropathy. Persons with diabetes are generally required to monitor their glucose levels to ensure that they are being maintained within a clinically safe range, and may also use this information to determine if and/or when insulin is needed to reduce glucose levels in their bodies, or when additional glucose is needed to raise the level of glucose in their bodies.

Growing clinical data demonstrates a strong correlation between the frequency of glucose monitoring and glycemic control. Despite such correlation, however, many individuals diagnosed with a diabetic condition do not monitor their glucose levels as frequently as they should due to a combination of factors including convenience, testing discretion, pain associated with glucose testing, and cost.

To increase patient adherence to a plan of frequent glucose monitoring, in vivo analyte monitoring systems can be utilized, in which a sensor control device may be worn on the body of an individual who requires analyte monitoring. To increase comfort and convenience for the individual, the sensor control device may have a small form-factor and can be applied by the individual with a sensor applicator. The application process includes inserting at least a portion of a sensor that senses a user's analyte level in a bodily fluid located in a layer of the human body, using an applicator or insertion mechanism, such that the sensor comes into contact with the bodily fluid. The analyte monitoring system may also be configured to transmit analyte data and/or alarms to another device, from which a caregiver such as, for example, a parent, a spouse, or a health care provider (“HCP”), can review the data and make therapy decisions. Furthermore, the benefits of analyte monitoring systems are not limited to persons with diabetes. For instance, analyte monitoring systems can provide useful information and insights to individuals interested in improving their health and wellness. As one example, to improve their sports performance, athletes can utilize a sensor control device worn on the body to collect data relating to one or more analytes such as, for example, glucose and/or lactate. Other non-medical applications for analyte monitoring systems are possible and described in further detail below.

Despite their advantages, however, some people are reluctant to use analyte monitoring systems for various reasons, including the complexity and volume of data presented, a learning curve associated with the software and user interfaces for analyte monitoring systems, and an overall paucity of actionable information presented.

Thus, needs exist for digital interfaces, graphical user interfaces, and alarms for analyte monitoring systems for medical and/or non-medical use, as well as methods and devices relating thereto, that are robust, user-friendly, and provide for timely and actionable responses.

### SUMMARY

Provided herein are example embodiments of digital and user interfaces for analyte monitoring systems. Aspects of the inventions are set out in the independent claims and preferred features are set out in the dependent claims. Preferred features of each aspect may be provided in combination with each other within particular embodiments and may also be provided in combination with other aspects.

Improved digital interfaces, graphical user interfaces, and alarms for analyte monitoring systems are provided. For example, disclosed herein are various embodiments of methods, systems, and interfaces for Silent Mode for alarms, Temporary Mode for alarms, escalating alarms, and alarm snooze features for an analyte monitoring software application. Also, various embodiments of interface enhancements are described, including caregiver alarms, among other embodiments. According to some embodiments, graphical user interfaces for enabling and disabling a nearby devices permission are provided to ensure that an analyte monitoring software application is configured properly and able to generate alarms.

According to another embodiment, enhancements to alarms for an analyte monitoring system software application are provided. In some embodiments, the alarm enhancement comprises a Silent Mode to allow the user to silence audible alarms for a predetermined period of time. In some embodiments, the alarm enhancement comprises a Temporary Mode to allow the user to temporarily silence audible alarms, temporarily output vibratory alerts for alarms, or temporarily output a maximum volume for alarms for a predetermined period of time. In some embodiments, the alarm enhancement comprises an escalating alarm to gradually increase the audible output of the alarm. In still other embodiments, the alarm enhancement is a snooze feature that allows the user to silence an alarm being presented for a predetermined period of time. In some embodiments, the alarm enhancement comprises a Back In Range feature which allows the user to be notified when the user has returned to a predetermined analyte range. In some embodiments, the user can customize sounds and vibration patterns for alarms.

According to another embodiment, methods, systems, and interfaces are provided for generating alarms relating to an analyte monitoring system on a caregiver's reader device. In some embodiments, for example, a sensor control device worn on a patient's body can wirelessly communicate current sensor readings to the patient's reader device which, in turn, can wirelessly communicate the current sensor readings to a cloud-based server. According to an aspect of the embodiments, the cloud-based server can determine whether the received current sensor readings satisfy an alarm condition and, if so, transmit an alarm indicator to the caregiver's reader device. In other embodiments, for example, an alarm condition is determined on the patient's reader device and, in response to the detection of the alarm condition, an alarm indicator is transmitted to the cloud-based server and subsequently “passed through” to the caregiver's reader device.

Many of the embodiments provided herein are improved GUIs or GUI features for analyte monitoring systems that are highly intuitive, user-friendly, and provide for rapid access to physiological information of a user. More specifically, these embodiments allow a user to easily navigate through and between different user interfaces that can quickly indicate to the user various physiological conditions and/or actionable responses, without requiring the user (or an HCP) to go through the arduous task of examining large volumes of analyte data. Furthermore, some of the GUIs and GUI features and interfaces allow for users (and their caregivers) to better understand and improve their respective levels of engagement with their analyte monitoring systems, and/or to troubleshoot complex system settings to ensure that alarms are functioning properly within an analyte monitoring system. Likewise, many other embodiments provided herein comprise improved digital interfaces, methods, and/or features for analyte monitoring systems that improve upon a caregiver's ability to determine an adverse condition of the patient. Other improvements and advantages are provided as well. The various configurations of these devices are described in detail by way of the embodiments which are only examples.

Other systems, devices, methods, features and advantages of the subject matter described herein will be or will become apparent to one with skill in the art upon examination of the following figures and detailed description. Where a method is described and claimed herein, analyte monitoring systems comprising means for performing each of the steps of the method are also expressly disclosed and provided. Moreover, computer programs, computer program products and computer readable media for implementing the steps of the method are also disclosed and provided. It is intended that all such additional systems, devices, methods, features, and advantages be included within this description, be within the scope of the subject matter described herein, and be protected by the accompanying claims. In no way should the features of the example embodiments be construed as limiting the appended claims, absent express recitation of those features in the claims.

### BRIEF DESCRIPTION OF THE FIGURES

The details of the subject matter set forth herein, both as to its structure and operation, may be apparent by study of the accompanying figures, in which like reference numerals refer to like parts. The components in the figures are not necessarily to scale, emphasis instead being placed upon illustrating the principles of the subject matter. Moreover, all illustrations are intended to convey concepts, where relative sizes, shapes and other detailed attributes may be illustrated schematically rather than literally or precisely.

FIG. 1 is a system overview of an analyte monitoring system comprising a sensor applicator, a sensor control device, a reader device, a network, a trusted computer system, and a local computer system.

FIG. 2A is a block diagram depicting an example embodiment of a reader device.

FIGS. 2B and 2C are block diagrams depicting example embodiments of sensor control devices.

FIG. 3A is a flow diagram depicting an example embodiment of a method for determining and presenting alarms in an analyte monitoring system.

FIGS. 3B to 3G are example embodiments of GUIs relating to various alarms in an analyte monitoring system.

FIG. 4A is a flow diagram depicting an example embodiment of a method for determining whether a nearby devices permission is enabled and modifying an analyte monitoring software application in response thereto.

FIGS. 4B to 4L are example embodiments of GUIs for an analyte monitoring software application in relation to a nearby devices permission.

FIGS. 5A to 5I are example embodiments of GUIs relating to various alarm settings in an analyte monitoring system.

FIGS. 5J to 5L are example embodiments of GUIs relating to alarm sound and vibration settings for alarms in an analyte monitoring system.

FIGS. 5M to 5N are example embodiments of GUIs relating to Back In Range settings and features related thereto in an analyte monitoring system.

FIGS. 6A to 6K-2 are example embodiments of GUIs relating to a Silent Mode for an analyte monitoring software application.

FIGS. 6L to 6Q are example embodiments of GUIs relating to a Temporary Mode for an analyte monitoring software application.

FIGS. 7A and 7B are example embodiments of GUIs relating to a snooze feature for an analyte monitoring software application.

FIG. 8 is a system overview of an analyte monitoring system comprising a sensor control device, a patient reader device, a network, a trusted computer system, and a caregiver reader device.

FIGS. 9A and 9B are flow diagrams depicting example embodiments of methods for providing caregiver alarms.

FIGS. 10A to 10H are example embodiments of GUIs relating to a caregiver application and alarm configuration settings included therewith.

### DETAILED DESCRIPTION

Before the present subject matter is described in detail, it is to be understood that this disclosure is not limited to the particular embodiments described, as such may, of course, vary. It is also to be understood that the terminology used herein is for the purpose of describing particular embodiments only, and is not intended to be limiting, since the scope of the present disclosure will be limited only by the appended claims.

As used herein and in the appended claims, the singular forms “a,” “an,” and “the” include plural referents unless the context clearly dictates otherwise.

The publications discussed herein are provided solely for their disclosure prior to the filing date of the present application. Nothing herein is to be construed as an admission that the present disclosure is not entitled to antedate such publication by virtue of prior disclosure. Further, the dates of publication provided may be different from the actual publication dates which may need to be independently confirmed.

Generally, embodiments of the present disclosure include GUIs, alarms, and digital interfaces for analyte monitoring systems, and systems, methods, and devices relating thereto. Accordingly, many embodiments include in vivo analyte sensors structurally configured so that at least a portion of the sensor is, or can be, positioned in the body of a user to obtain information about at least one analyte of the body. It should be noted, however, that the embodiments disclosed herein can be used with in vivo analyte monitoring systems that incorporate in vitro capability, as well as purely in vitro or ex vivo analyte monitoring systems, including systems that are entirely non-invasive.

Furthermore, for each and every embodiment of a method disclosed herein, systems and devices capable of performing each of those embodiments are covered within the scope of the present disclosure. For example, embodiments of sensor control devices, reader devices, local computer systems, and trusted computer systems are disclosed, and these devices and systems can have one or more sensors, analyte monitoring circuits (e.g., an analog circuit), memories (e.g., for storing instructions), power sources, communication circuits, transmitters, receivers, processors and/or controllers (e.g., for executing instructions) that can perform any and all method steps or facilitate the execution of any and all method steps.

As previously described, a number of embodiments described herein provide for improved GUIs, alarms, and digital interfaces for analyte monitoring systems, wherein the alarms and GUIs are actionable, user-friendly, and provide for rapid access to physiological information of a user. According to some embodiments, for example, methods and interfaces are provided for enhancement to alarms for an analyte monitoring software application. According to some embodiments, for example, methods and interfaces are provided for determining an alarm unavailability condition in an analyte monitoring system due to a disabled nearby devices permission. Additional improved digital and user interfaces for an analyte monitoring software application are described.

According to another embodiment, methods, systems, and interfaces are provided for generating alarms relating to an analyte monitoring system on a caregiver's reader device. In some embodiments, for example, a sensor control device worn on a patient's body can wirelessly communicate current sensor readings to the patient's reader device which, in turn, can wirelessly communicate the current sensor readings to a cloud-based server. According to an aspect of the embodiments, the cloud-based server can determine whether the received current sensor readings satisfy an alarm condition and, if so, transmit an alarm indicator to the caregiver's reader device. In other embodiments, for example, an alarm condition is determined on the patient's reader device and, in response to the detection of an alarm condition, an alarm indicator is transmitted to the cloud-based server and subsequently “passed through” to the caregiver's reader device.

Collectively and individually, these methods, systems, and digital and user interfaces improve upon the accuracy, integrity, and privacy of analyte data being collected by an analyte monitoring system, the flexibility of the analyte monitoring system by allowing caregivers to receive information about a patient's condition, and the alarming capabilities of the analyte monitoring system. Other improvements and advantages are provided as well. The various configurations of these devices are described in detail by way of the embodiments which are only examples.

Before describing these aspects of the embodiments in detail, however, it is first desirable to describe examples of devices that can be present within, for example, an in vivo analyte monitoring system, as well as examples of their operation, all of which can be used with the embodiments described herein.

There are various types of in vivo analyte monitoring systems. “Continuous Analyte Monitoring” systems (or “Continuous Glucose Monitoring” systems), for example, can transmit data from a sensor control device to a reader device continuously without prompting, e.g., automatically according to a schedule. “Flash Analyte Monitoring” systems (or “Flash Glucose Monitoring” systems or simply “Flash” systems), as another example, can transfer data from a sensor control device in response to a scan or request for data by a reader device, such as with a Near Field Communication (NFC) or Radio Frequency Identification (RFID) protocol. In vivo analyte monitoring systems can also operate without the need for finger stick calibration.

In vivo analyte monitoring systems can be differentiated from “in vitro” systems that contact a biological sample outside of the body (or “ex vivo”) and that typically include a meter device that has a port for receiving an analyte test strip carrying bodily fluid of the user, which can be analyzed to determine the user's blood sugar level.

In vivo monitoring systems can include a sensor that, while positioned in vivo, makes contact with the bodily fluid of the user and senses the analyte levels contained therein. The sensor can be part of the sensor control device that resides on the body of the user and contains the electronics and power supply that enable and control the analyte sensing. The sensor control device, and variations thereof, can also be referred to as a “sensor control unit,” an “on-body electronics” device or unit, an “on-body” device or unit, or a “sensor data communication” device or unit, to name a few.

In vivo monitoring systems can also include a device that receives sensed analyte data from the sensor control device and processes and/or displays that sensed analyte data, in any number of forms, to the user. This device, and variations thereof, can be referred to as a “handheld reader device,” “reader device” (or simply a “reader”), “handheld electronics” (or simply a “handheld”), a “portable data processing” device or unit, a “data receiver,” a “receiver” device or unit (or simply a “receiver”), or a “remote” device or unit, to name a few. Other devices such as personal computers have also been utilized with or incorporated into in vivo and in vitro monitoring systems.

### Example Embodiment of In Vivo Analyte Monitoring System

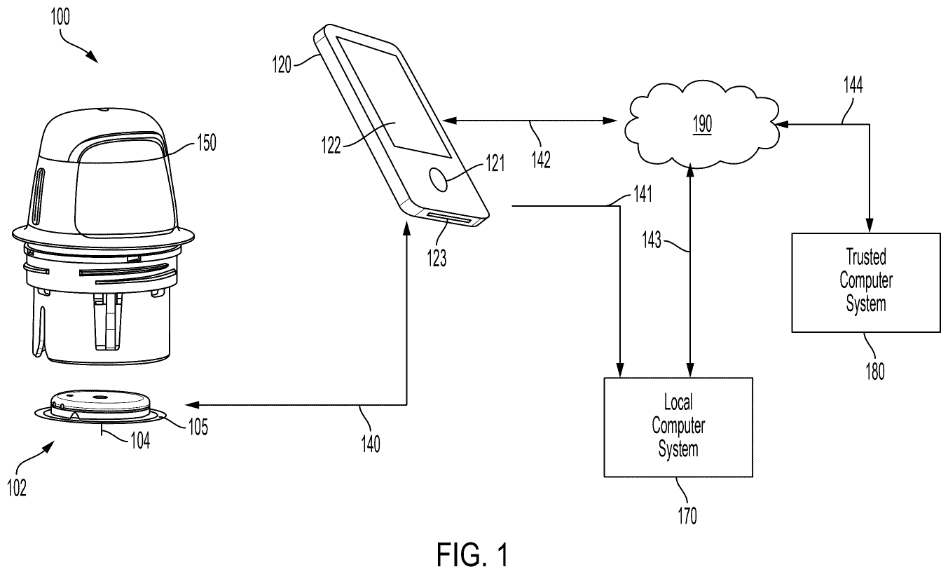

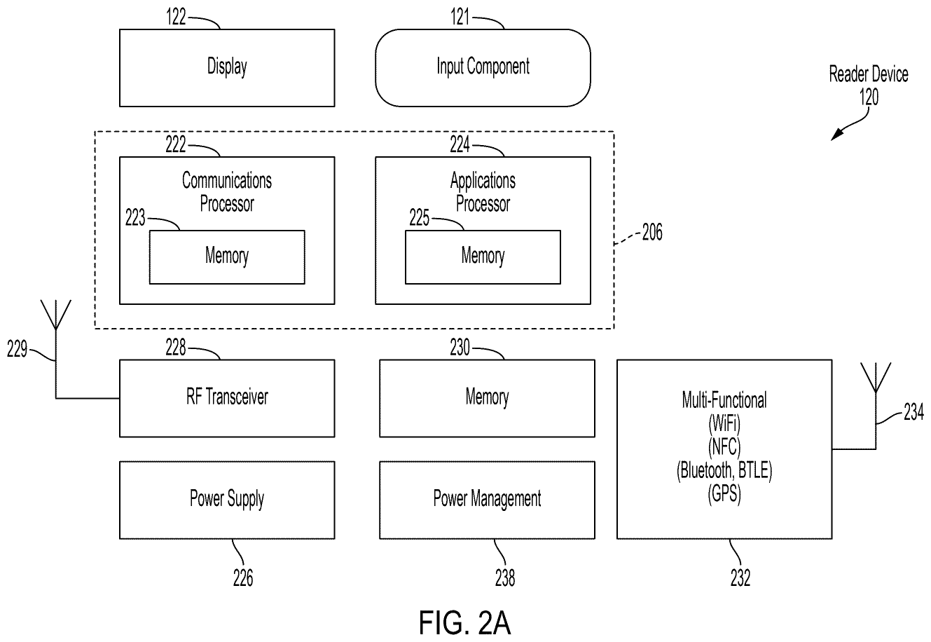

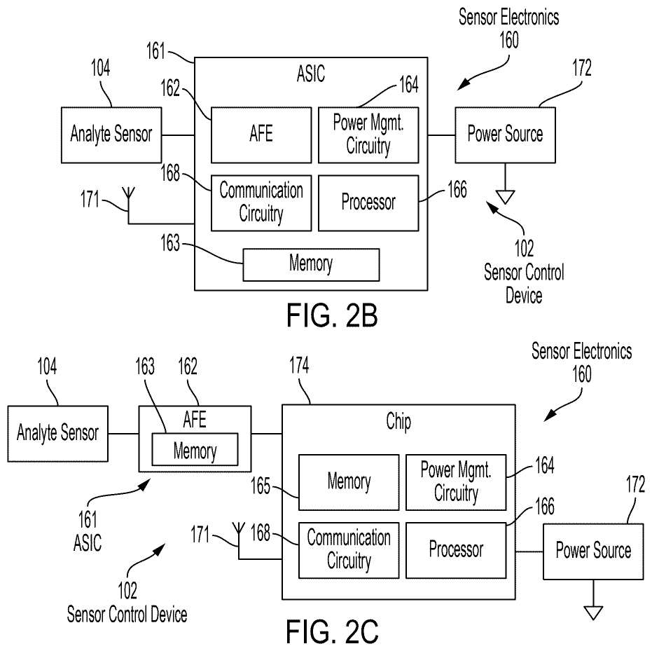

FIG. 1 is a conceptual diagram depicting an example embodiment of an analyte monitoring system 100 that includes a sensor applicator 150, a sensor control device 102, and a reader device 120. Here, sensor applicator 150 can be used to deliver sensor control device 102 to a monitoring location on a user's skin where a sensor 104 is maintained in position for a period of time by an adhesive patch 105. Sensor control device 102 is further described in FIGS. 2B and 2C, and can communicate with reader device 120 via a communication path 140 using a wired or wireless technique. Example wireless protocols include Bluetooth, Bluetooth Low Energy (BLE, BTLE, Bluetooth SMART, etc.), Near Field Communication (NFC) and others. Users can view and use applications installed in memory on reader device 120 using screen 122 (which, in many embodiments, can comprise a touchscreen), and input 121. A device battery of reader device 120 can be recharged using power port 123. While only one reader device 120 is shown, sensor control device 102 can communicate with multiple reader devices 120. Each of the reader devices 120 can communicate and share data with one another. More details about reader device 120 is set forth with respect to FIG. 2A below. Reader device 120 can communicate with local computer system 170 via a communication path 141 using a wired or wireless communication protocol. Local computer system 170 can include one or more of a laptop, desktop, tablet, phablet, smartphone, set-top box, video game console, or other computing device and wireless communication can include any of a number of applicable wireless networking protocols including Bluetooth, Bluetooth Low Energy (BTLE), Wi-Fi or others. Local computer system 170 can communicate via communications path 143 with a network 190 similar to how reader device 120 can communicate via a communications path 142 with network 190, by a wired or wireless communication protocol as described previously. Network 190 can be any of a number of networks, such as private networks and public networks, local area or wide area networks, and so forth. A trusted computer system 180 can include a cloud-based platform or server, and can provide for authentication services, secured data storage, report generation, and can communicate via communications path 144 with network 190 by wired or wireless technique. In addition, although FIG. 1 depicts trusted computer system 180 and local computer system 170 communicating with a single sensor control device 102 and a single reader device 120, it will be appreciated by those of skill in the art that local computer system 170 and/or trusted computer system 180 are each capable of being in wired or wireless communication with a plurality of reader devices and sensor control devices.

### Example Embodiment of Reader Device

FIG. 2A is a block diagram depicting an example embodiment of a reader device 120, which, in some embodiments, can comprise a smart phone. Here, reader device 120 can include a display 122, input component 121, and a processing core 206 including a communications processor 222 coupled with memory 223 and an applications processor 224 coupled with memory 225. Also included can be separate memory 230, RF transceiver 228 with antenna 229, and power supply 226 with power management module 238. Further, reader device 120 can also include a multi-functional transceiver 232, which can comprise wireless communication circuitry, and which can be configured to communicate over Wi-Fi, NFC, Bluetooth, BTLE, and GPS with an antenna 234. As understood by one of skill in the art, these components are electrically and communicatively coupled in a manner to make a functional device.

### Example Embodiments of Sensor Control Devices

FIGS. 2B and 2C are block diagrams depicting example embodiments of sensor control devices 102 having analyte sensors 104 and sensor electronics 160 (including analyte monitoring circuitry) that can have the majority of the processing capability for rendering end-result data suitable for display to the user. In FIG. 2B, a single semiconductor chip 161 is depicted that can be a custom application specific integrated circuit (ASIC). Shown within ASIC 161 are certain high-level functional units, including an analog front end (AFE) 162, power management (or control) circuitry 164, processor 166, and communication circuitry 168 (which can be implemented as a transmitter, receiver, transceiver, passive circuit, or otherwise according to the communication protocol). In this embodiment, both AFE 162 and processor 166 are used as analyte monitoring circuitry, but in other embodiments either circuit can perform the analyte monitoring function. Processor 166 can include one or more processors, microprocessors, controllers, and/or microcontrollers, each of which can be a discrete chip or distributed amongst (and a portion of) a number of different chips.

A memory 163 is also included within ASIC 161 and can be shared by the various functional units present within ASIC 161, or can be distributed amongst two or more of them. Memory 163 can also be a separate chip. Memory 163 can be volatile and/or non-volatile memory. In this embodiment, ASIC 161 is coupled with power source 172, which can be a coin cell battery, or the like. AFE 162 interfaces with in vivo analyte sensor 104 and receives measurement data therefrom and outputs the data to processor 166 in digital form, which in turn processes the data to arrive at the end-result glucose discrete and trend values, etc. This data can then be provided to communication circuitry 168 for sending, by way of antenna 171, to reader device 120 (not shown), for example, where minimal further processing is needed by the resident software application to display the data. According to some embodiments, for example, a current glucose value can be transmitted from sensor control device 102 to reader device 120 every minute, and historical glucose values can be transmitted from sensor control device 102 to reader device 120 every five minutes.

In some embodiments, data acquired from sensor control device 102 can be stored on reader device 120. According to one aspect of some embodiments, such data can include the model number and serial number of sensor control device 102, as well as information relating to the sensor control device 102's status, market code, or network address. In some embodiments, such data can also include error events detected by sensor control device 102. In addition, in some embodiments, either or both of current glucose values and historical glucose values can include one or more time stamps (e.g., factory time, UTC time, user's local time based on time zone, and the current time zone).

In some embodiments, sensor control device 102 can store data such that if reader device 120 is not in communication with sensor control device 102 (e.g., if reader device 120 is out of a wireless communication range, is powered off, or is otherwise unable to communicate with sensor control device 102), when reader device 120 re-establishes communication with sensor control device 102, data can then be backfilled to reader device 120. According to some embodiments, data that can be backfilled can include, but is not limited to, current and historical glucose values, as well as error events. Further details regarding data backfilling can be found in U.S. Pat. No. 10,820,842, as well as U.S. Publ. No. 2021/0282672 (“the '672 Publication”), both of which are hereby incorporated by reference in their entireties for all purposes.

According to some embodiments, each current glucose value and/or historical glucose value acquired from sensor control device 102 can further be validated on reader device 120, such as, for example, by performing a CRC integrity check to ensure that the data has been transferred accurately. In some embodiments, for example, a data quality mask of the current glucose value and/or historical glucose value can be checked to ensure that the reading is correct and can be displayed as a valid reading on the reader device 120.

According to another aspect of some embodiments, reader device 120 can include a database for storing any or all of the aforementioned data. In some embodiments, the database can be configured to retain data for a predetermined period of time (e.g., 30 days, 60 days, 90 days, six months, one year, etc.). According to some embodiments, the database can be configured to delete data after it has been uploaded to a cloud server. In other embodiments, the database can be configured for a clinical setting, in which data is retained for a longer period of time (e.g., one year) relative to a non-clinical setting. In addition to the aforementioned data (e.g., current and/or historical glucose values, error events, etc.), the database on reader device 120 can also store user configuration information (e.g., login ID, notification settings, regional settings, and other preferences), as well as application configuration information (e.g., cloud settings, URLs for uploading data and/or error events, version information, etc.). The database can be encrypted to prevent a user from inspecting the data content directly even if the operating system of reader device 120 is compromised.

In some embodiments, to conserve power and processing resources on sensor control device 102, digital data received from AFE 162 can be sent to reader device 120 (not shown) with minimal or no processing. In still other embodiments, processor 166 can be configured to generate certain predetermined data types (e.g., current glucose value, historical glucose values) either for storage in memory 163 or transmission to reader device 120 (not shown), and to ascertain certain alarm conditions (e.g., sensor fault conditions), while other processing and alarm functions (e.g., high/low glucose threshold alarms) can be performed on reader device 120. Those of skill in the art will understand that the methods, functions, and interfaces described herein can be performed—in whole or in part—by processing circuitry on sensor control device 102, reader device 120, local computer system 170, or trusted computer system 180.

FIG. 2C is similar to FIG. 2B but instead includes two discrete semiconductor chips 162 and 174, which can be packaged together or separately. Here, AFE 162 is resident on ASIC 161. Processor 166 is integrated with power management circuitry 164 and communication circuitry 168 on chip 174. AFE 162 may include memory 163 and chip 174 includes memory 165, which can be isolated or distributed within. In one example embodiment, AFE 162 is combined with power management circuitry 164 and processor 166 on one chip, while communication circuitry 168 is on a separate chip. In another example embodiment, both AFE 162 and communication circuitry 168 are on one chip, and processor 166 and power management circuitry 164 are on another chip. It should be noted that other chip combinations are possible, including three or more chips, each bearing responsibility for the separate functions described, or sharing one or more functions for fail-safe redundancy.

### Example Embodiments of Graphical User Interfaces for Analyte Monitoring Systems

Described herein are example embodiments of GUIs for analyte monitoring systems, as well as methods and system relating thereto. As an initial matter, it will be understood by those of skill in the art that the GUIs and related methods described herein comprise instructions stored in a non-transitory memory of reader device 120, local computer system 170, trusted computer system 180, and/or any other device or system that is part of, or in communication with, analyte monitoring system 100. These instructions, when executed by one or more processors of the reader device 120, local computer system 170, trusted computer system 180, or other device or system of analyte monitoring system 100, cause the one or more processors to perform the method steps and/or output the GUIs described herein. Those of skill in the art will further recognize that the GUIs described herein can be stored as instructions in the non-transitory memory of a single centralized device or, in the alternative, can be distributed across multiple discrete devices in geographically dispersed locations.

Also described herein are example embodiments of alarms, alarm features, and alarm settings for analyte monitoring systems, as well as methods, systems, and GUIs relating thereto. As an initial matter, it will be understood by those of skill in the art that the alarms, alarm features, and alarm settings, as well as the related methods and interfaces described herein, can comprise instructions (e.g., software, firmware, etc.) stored in non-transitory memory of a sensor control device 102, reader device 120, local computer system 170, trusted computer system 180, and/or any other computing device or system (e.g., cloud-based server) that is part of, or in communication with, analyte monitoring system 100. These instructions, when executed by one or more processors of the corresponding system or device, cause the one or more processors to perform any or all of the method steps, and/or output the alarms or alarm interfaces described herein. Those of skill in the art will further recognize that the alarms, alarm features, alarm settings, and alarm interfaces described herein can be stored as instructions in the non-transitory memory of a single centralized device or, in the alternative, can be distributed across multiple discrete devices in geographically dispersed locations.

### Example Embodiments of Signal Loss Indicators, Alarms, and Other Errors Conditions

Example embodiments of methods and systems for determining a signal loss condition, generating signal loss indicators and alarms, and outputting signal loss interfaces and GUIs will now be described. According to one aspect of the embodiments, a signal loss condition can refer to a state of a device capable of wireless communication in an analyte monitoring system, such as a reader device (e.g., smart phone), sensor control unit, or local computing system, in which the device has not received a current sensor reading for a predetermined period of time. According to another aspect of the embodiments, a signal loss alarm condition can comprise a state in which the device has not received a valid current sensor reading for another predetermined period of time. In some embodiments, an invalid current sensor reading can comprise one of a failure to receive a current sensor reading within a predetermined time period, an error relating to a sensor signal, or an error relating to the temperature of the sensor.

### Example Embodiments of Methods and Systems for Enhanced Alarm Interfaces

Various example embodiments relating to alarming and alarm settings interfaces, for analyte monitoring systems, and other related features will now be described. It will be understood by those of skill in the art that any one or more of the example embodiments of the methods, interfaces, and systems described herein can either be implemented independently, or in combination with any of the other embodiments described in the present application.

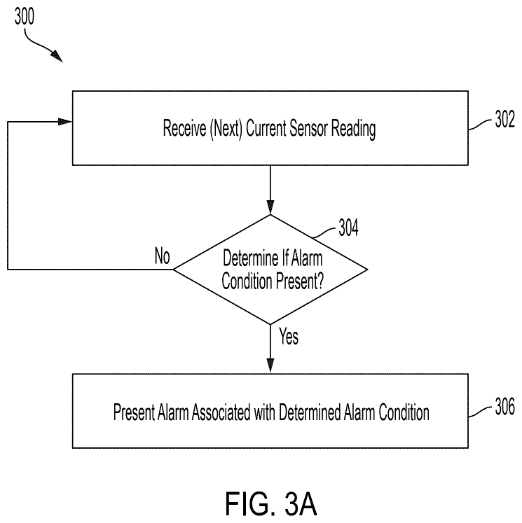

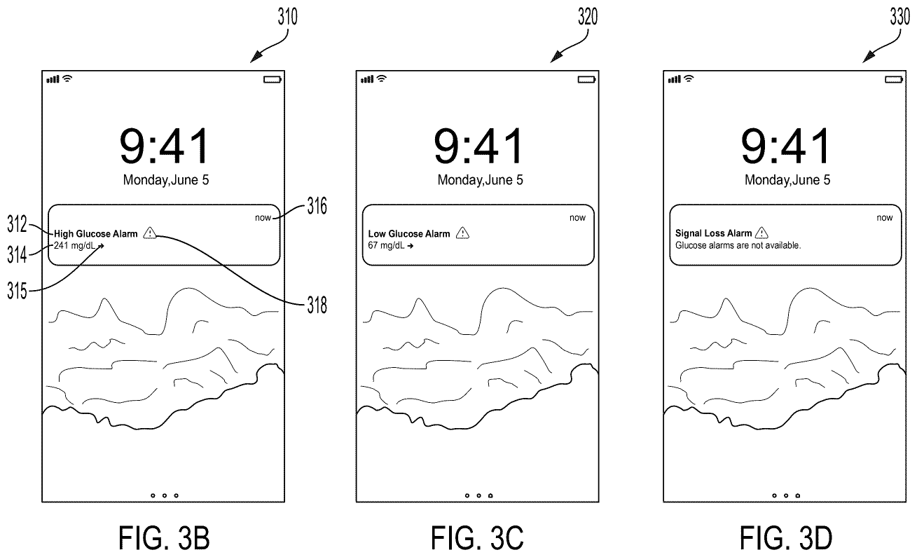

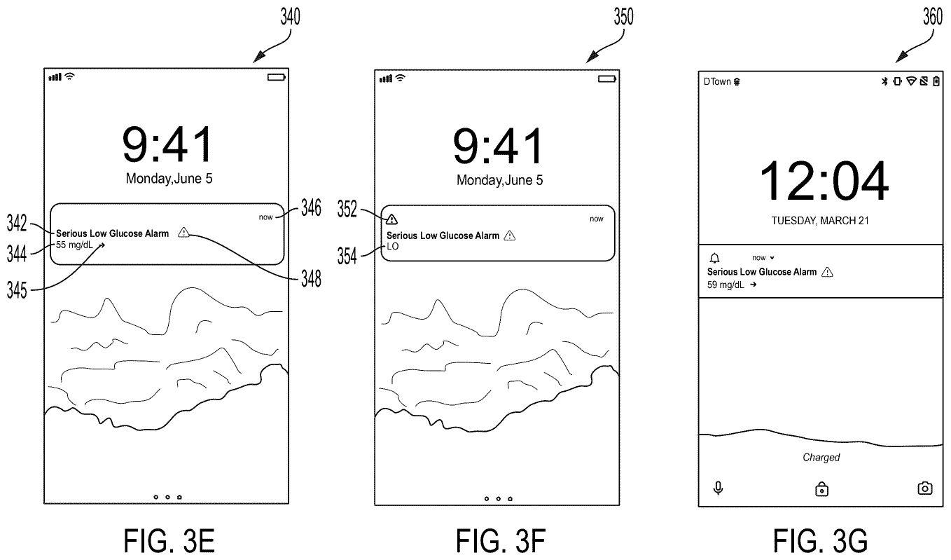

FIG. 3A depicts an example embodiment of a method 300 for determining one or more alarm conditions and presenting an alarm associated with the determined one or more alarm conditions. At Step 302, a current sensor reading is received. In some embodiments, the current sensor reading can comprise one or more signals from a glucose sensor disposed in the sensor control device 102, wherein at least a portion of the glucose sensor is configured to be positioned under the subject's skin and in contact with a bodily fluid. In other embodiments, the current sensor reading can be a glucose level measurement received by reader device 120. In some embodiments, the reader device 120 can be in direct communication with sensor control device 102. In other embodiments, reader device 120 can receive a glucose level measurement via another computing device, such as, for example, a cloud-based server. At Step 304, a determination is made as to whether one or more alarm conditions are present. According to some embodiments, for example, the one or more alarm conditions can comprise at least one of: a low glucose condition, an urgent low glucose condition (also sometimes referred to as a “serious low glucose condition” or a “fixed low glucose condition”), a high glucose condition, a rapidly dropping glucose (rate-of-change) condition, a rapidly rising glucose (rate-of-change) condition, a predicted low glucose condition, or a predicted high glucose condition, amongst other alarm conditions. In some embodiments, the alarm condition can also comprise a signal loss condition, wherein a valid current glucose reading has not been received within a predetermined amount of time (e.g., one minute, five minutes, ten minutes, twenty minutes, etc.). In some embodiments, the signal loss condition can be a result of a loss of wireless connectivity (e.g., Bluetooth connectivity) between reader device 120 and sensor control device 102. As stated earlier, the determination step can be performed by reader device 120, sensor control device 102, or any another computing device described with respect to analyte monitoring system 100.

Referring still to FIG. 3A, at Step 306, if it is determined that the one or more alarm conditions are present, an alarm associated with the determined alarm condition is presented. In some embodiments, the presentation of the alarm can comprise a visual notification (e.g., pop-up window, banner notification, full screen notification, etc.). In other embodiments, the presentation of the alarm can comprise a visual notification accompanied by an audible and/or vibratory indicator. In still other embodiments, the presentation of the alarm can comprise an audible and/or vibratory indicator, without a visual notification.

It will be appreciated by those of skill in the art that the method steps described herein can be performed either by a single device or by multiple devices. For example, in some embodiments, the determination of the one or more alarm conditions can be performed by sensor control device 102 and the presentation of alarms can be performed by reader device 120. In other embodiments, both the determination of the alarm condition and the presentation of the alarm can be performed by reader device 120.

FIGS. 3B to 3D are example embodiments of GUIs comprising alarms for use with an analyte monitoring system. FIG. 3B, for example, is a GUI 310 depicting an alarm for an analyte monitoring system, wherein the alarm comprises an alarm condition text 312 (e.g., “High Glucose Alarm”), an analyte level measurement 314 (e.g., a current glucose level of 241 mg/dL) associated with the alarm condition, and a trend indicator 315 (e.g., a trend arrow or directional arrow) associated with the alarm condition. Additionally, a time-of-alarm indicator 316 is also shown. In some embodiments, the time-of-alarm indicator 316 can indicate the amount of time elapsed since the alarm condition was triggered (e.g., now, 5 min ago, 10 min ago). In other embodiments, the time-of-alarm indicator 316 can indicate the specific time of that the alarm condition was triggered (e.g., 6:12 p.m.). In some embodiments, an alarm icon 318 can also be adjacent to the alarm condition text 312.

FIG. 3C is a GUI 320 depicting another alarm for an analyte monitoring system, wherein the alarm comprises a low glucose alarm for a low glucose alarm condition. FIG. 3D is a GUI 330 depicting another alarm for an analyte monitoring system, wherein the alarm comprises a signal loss alarm for a signal loss condition.

### Example Embodiments of Urgent Low Glucose Alarms

Example embodiments of urgent low glucose alarms will now be described. In a general sense, urgent low glucose alarms for analyte monitoring systems share certain similarities to the alarms previously described with respect to FIGS. 3B to 3D. According to one aspect of the embodiments, urgent low glucose alarms will present an alarm to the user when his or her glucose level has fallen below an urgent low glucose threshold (e.g., below 55 mg/dL). According to another aspect of the embodiments, because of the critical nature of the user's condition, urgent low glucose alarms will override other settings of reader device 120, including the reader device's operating system, such as a “Mute” or “Do Not Disturb” setting. Further, in many embodiments, the glucose threshold levels, which are typically adjustable for other alarms, cannot be changed for urgent low glucose alarms.

FIGS. 3E to 3G are example embodiments of GUIs comprising urgent low glucose alarms for use in an analyte monitoring system. As stated above, these embodiments of alarms have some similarities to the embodiments depicted in FIGS. 3B to 3D. FIG. 3E, for example, is a GUI 340 depicting an urgent low glucose alarm for an analyte monitoring system, wherein the alarm comprises an urgent low glucose alarm condition text 342, an analyte level measurement 344 (e.g., 55 mg/dL) associated with the alarm condition, and a trend indicator 345 (e.g., a trend arrow or directional arrow) associated with the alarm condition. Additionally, a time-of-alarm indicator 346 and an alarm icon 348 are also shown. FIG. 3F is another GUI 350 depicting an urgent low glucose alarm. As seen in FIG. 3F, a critical alert icon 352 and an out-of-range (“LO”) text indicator 354 are displayed in GUI 350, indicating that the glucose level has dropped below a minimum threshold value within a measurable range. FIG. 3G is another GUI 360 depicting an urgent low glucose alarm similar to embodiments previously described, but presented through a different mobile operating system (e.g., Android) than the embodiment shown in FIGS. 3E and 3F (e.g., iOS).

According to an aspect of these embodiments, the alarms described herein can include both alarms with configurable settings (e.g., low glucose alarm, high glucose alarm, signal loss alarm) and alarms with non-configurable settings (e.g., urgent low glucose alarm) operating on a single computing device within the same analyte monitoring system. In some embodiments, for example, an analyte monitoring system can include a reader device comprising wireless communication circuitry configured to receive data indicative of an analyte level from a sensor control device, and one or more processors coupled with a memory, the memory storing instructions that, when executed by the one or more processors, cause the one or more processors to: (1) determine whether the data indicative of the analyte level meets one or more alarm conditions, wherein the one or more alarm conditions comprises a first alarm condition associated with a first set of alarms settings that are configurable by the subject, and a second alarm condition associated with a second set of alarm settings that are not configurable by the subject, and wherein the second alarm condition is an urgent low glucose alarm condition; and (2) in response to a determination that at least one of the one or more alarm conditions is met, present an alarm associated with the at least one of the one or more alarm conditions.

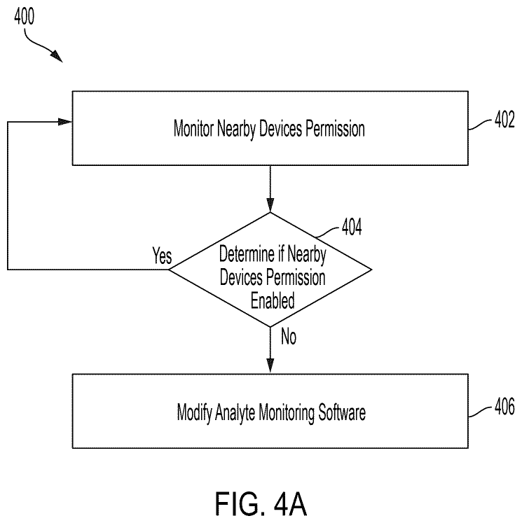

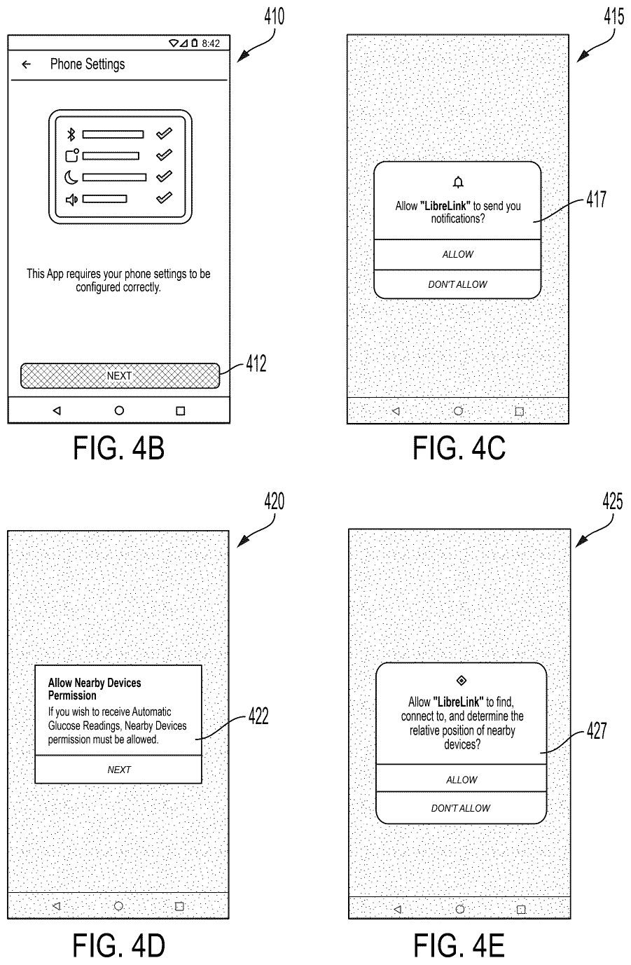

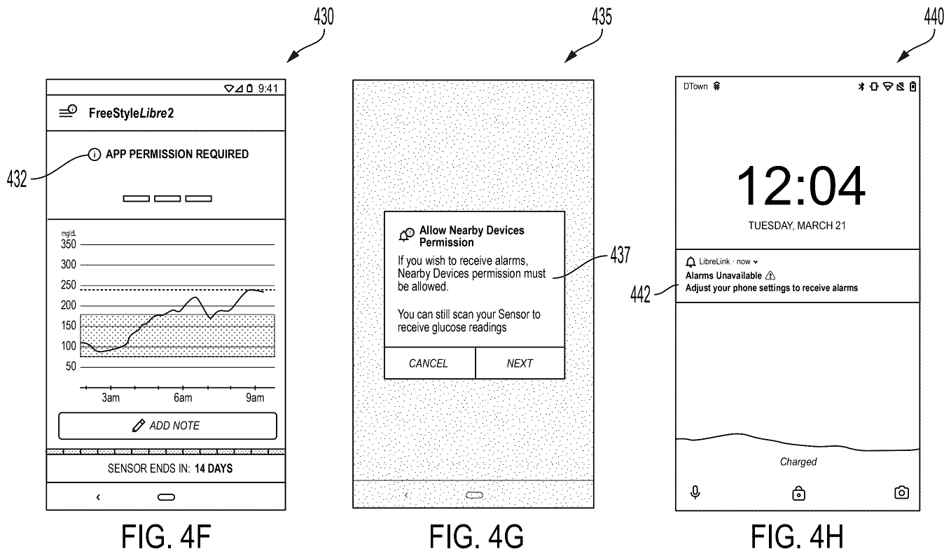

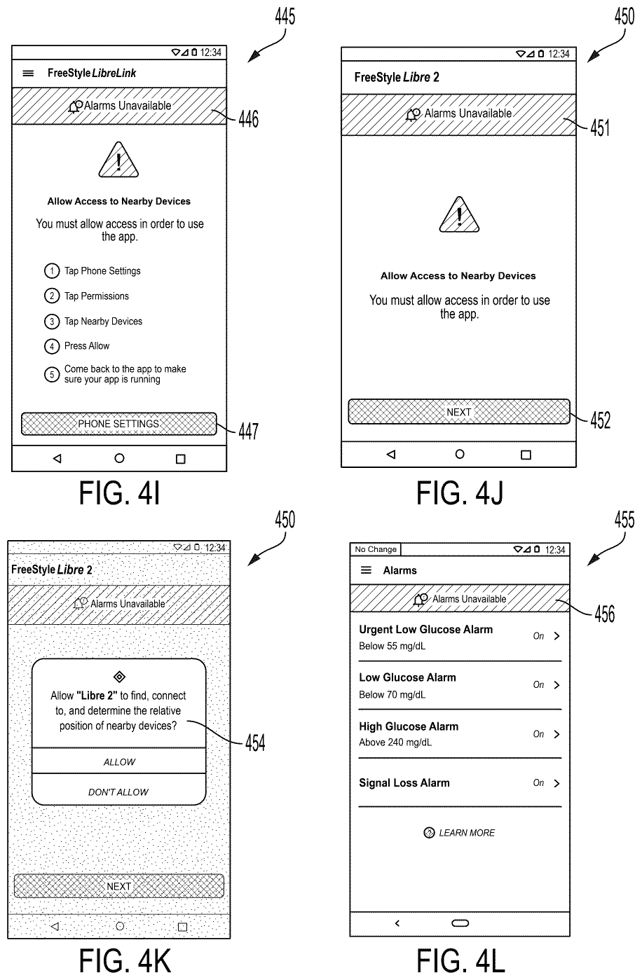

FIG. 4A depicts an example embodiment of a method 400 for monitoring a nearby devices permission and modifying an analyte monitoring software program in response to determining that the nearby devices permission is disabled. In some embodiments, an operating system of a reader device may include a nearby devices permission that determines whether an application installed on the reader device can find, connect to, and/or determine the position of other nearby devices. More specifically, granting an application with the nearby devices permission allows the application to search for nearby devices and networks using wireless communication protocols (e.g., Wi-Fi) that are separate from broader location services and/or navigational systems (e.g., GPS).

Referring to FIG. 4A, at Step 402, a nearby devices permission is monitored by the analyte monitoring software program. In some embodiments, this can be a periodic routine performed by the analyte monitoring software program while it is running (e.g., every 5 minutes, every 10 minutes, every 15 minutes, etc.). In some embodiments, this can be a routine performed by the analyte monitoring software program when it is first started or, in the alternative, in response to a predetermined event (e.g., viewing an alarms settings GUI) or set of predetermined events (e.g., navigating from one GUI to another).

At Step 404, during the monitoring of the nearby devices permission, a determination is made as to whether the nearby devices permission is enabled for the analyte monitoring software program. If the permission is enabled, then the analyte monitoring software program continues to operate. If the nearby devices permission is disabled then, at Step 406, the analyte monitoring software program is modified in response thereto. In some embodiments, for example, the modification can be a blocking of the analyte monitoring software program such that no function or feature of the analyte monitoring software program is made available to the user until the nearby device's permission is enabled. In some embodiments, the modification can comprise an indication by the analyte monitoring software program that alarms are unavailable. In said embodiments, certain functions and/or features of the analyte monitoring software program can still be available to the user. See FIG. 4F. Example embodiments of graphical user interfaces (GUIs) for an analyte monitoring software program relating to such modifications are shown in FIGS. 4F to 4L.

Referring next to FIGS. 4B to 4L, various example embodiments of graphical user interfaces relating to the nearby devices setting are depicted and will now be described.

FIG. 4B is an example embodiment of an informational GUI 410 for an analyte monitoring software program, wherein GUI 410 indicates to the user that the analyte monitoring software program requires certain settings to be configured correctly in order for the analyte monitoring software program to operate properly. According to some embodiments, GUI 400 can include a “Next” button such that the information presented remains on the screen until the user affirmatively acknowledges reading the screen by pressing the button.

FIG. 4C is an example embodiment of a GUI 415 for an analyte monitoring software program, wherein GUI 415 includes a modal 417 for prompting the user to select an option to either allow or disallow the analyte monitoring software program from sending notifications. The term “modal,” as used herein, can include but is not limited to a window, a notification, or pop-up that overlays, partially overlays, or appears on an underlying GUI so as to allow the underlying GUI to remain at least partially visible. According to many embodiments, the notifications setting is another permission granted by the operating system of the reader device that is separate from the nearby devices permission. In this regard, according to many embodiments, selection of the “allow” option enables a notifications permission of the operating system to allow the analyte monitoring software program to send notifications to the user.

FIG. 4D is an example embodiment of a GUI 420 for an analyte monitoring software program, wherein GUI 420 includes a modal 422 to inform the user that, to wirelessly receive data indicative of a glucose level (e.g., glucose readings) from the sensor control device, the nearby devices permission must be enabled or allowed. Modal 422 can include a “Next” button such that the information presented remains on the screen until the user affirmatively acknowledges reading the screen by pressing the button.

FIG. 4E is an example embodiment of a GUI 425 for an analyte monitoring software program, wherein GUI 425 includes a modal 427 for prompting the user to either allow or disallow the nearby devices permission with respect to the analyte monitoring software program. As described earlier, in some embodiments, the nearby devices permission allows the application to find, connect to, and determine the relative position of nearby devices. In this regard, according to many embodiments, selection of the “allow” option enables the nearby devices permission of the operating system with respect to the analyte monitoring software program.

Referring next to FIG. 4F, an example embodiment of a GUI 430 for an analyte monitoring software program is depicted. According to some embodiments, upon determination that the nearby devices permission is not enabled, the analyte monitoring software application can be modified in response thereto. In some embodiments, for example, a sensor results GUI 430 can be modified such that a banner notification 432 is displayed on the screen indicating that a particular permission is required. According to some embodiments, GUI 430 can continue to display the user's historical glucose data even though the nearby devices permission has been determined to be disabled. However, as can be seen at top of GUI 430, a numeric current glucose value can be replaced by banner notification 432 and three dashed lines.

Referring still to FIG. 4F, banner notification 432 can include a selectable informational icon that, when selected by the user, navigates the user to a different GUI 435, as shown in FIG. 4G. According to some embodiments, GUI 435 is similar to GUI 420 (FIG. 4D), in that it includes a modal 437 to inform the user that the nearby devices permission must be enabled or allowed for the analyte monitoring software program to receive alarms. Likewise, modal 437 can include a “Next” button and/or a “Cancel” button, such that the information presented remains on the screen until the user affirmatively acknowledges the modal 437 by pressing one of the buttons. According to many embodiments, selection of the “Next” button can navigate the user to another GUI, such as GUI 425 (FIG. 4E).

Turning next to FIG. 4H, an example embodiment of an alert GUI 440 for an analyte monitoring software program is depicted. According to one aspect of some embodiments, upon determination that the nearby devices permission is not enabled, the analyte monitoring software application can be modified in response thereto. In some embodiments, for example, an alert GUI 440 can be displayed on a lock screen of the reader device, such as in case the analyte monitoring software application is not running in the foreground. In some embodiments, alert GUI 440 can indicate to the user that alarms are unavailable, and that the user is required to adjust her phone settings to receive alarms. According to some embodiments, selection of the alert GUI 440 can navigate the user to another GUI, such as GUI 425 (FIG. 4E).

Referring next to FIG. 4I, an example embodiment of an informational blocked app GUI 445 for an analyte monitoring software application is depicted. According to some embodiments, upon determination that the nearby devices permission is not enabled, the analyte monitoring software application can be blocked in response thereto. In some embodiments, for example, an informational blocked app GUI 445 can be presented in response to the determination that the nearby devices permission is not enabled, wherein the informational blocked app GUI 445 can indicate to the user a series of instructions regarding how to manually enable the nearby devices permission. In some embodiments, an in-app banner notification 446 can also be displayed to indicate that alarms are unavailable. Further operation of the analyte monitoring software application is prevented until the user enables the nearby devices permission. In some embodiments, the informational blocked app GUI 445 can further include a button 447 that, when selected, navigates the user to another GUI configured to allow the user to enable the nearby devices permission, such as a settings GUI that is part of the operating system of the reader device.

Turning next to FIGS. 4J and 4K, another example embodiment of an informational blocked app GUI 450 for an analyte monitoring software application is depicted. As described above, according to some embodiments, upon determination that the nearby devices permission is not enabled, the analyte monitoring software application can be blocked in response thereto, which prevents the user from further operation of the analyte monitoring software application until the nearby device's permission is enabled. In some embodiments, an in-app banner notification 451 can also be displayed to indicate that alarms are unavailable. In some embodiments, the informational blocked app GUI 450 can include a “Next” button 452 which, when selected, displays a modal 454 (FIG. 4K). According to many embodiments, modal 454, like modal 427 (FIG. 4E), prompts the user to either allow or disallow the nearby devices permission with respect to the analyte monitoring software program. According to many embodiments, selection of the “allow” option enables the nearby devices permission of the operating system with respect to the analyte monitoring software program.

Referring next to FIG. 4L, an example embodiment of an alarm settings GUI 455 for an analyte monitoring software application is depicted. As described above, according to some embodiments, upon determination that the nearby devices permission is not enabled, the analyte monitoring software application can be modified in response thereto. In some embodiments, for example, an alarm settings GUI 455 can be modified to display an in-app banner notification 456 that alarms are unavailable. In some embodiments, alarm settings GUI 455 can be further modified to prevent the changing of one or more alarm settings such as, for example, enabling or disabling a low glucose alarm, changing a low glucose threshold value, enabling or disabling a high glucose alarm, changing a high glucose threshold value, and/or enabling or disabling a signal loss alarm. Those of skill in the art will appreciate that these aforementioned alarm settings are merely illustrative and non-exhaustive, and that changing other alarm settings can be prevented in response to a determination that the nearby devices permission is not enabled.

Example embodiments of alarm enhancements and alarm settings for an analyte monitoring software application will now be described.

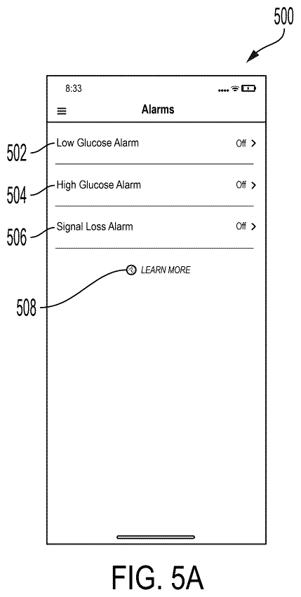

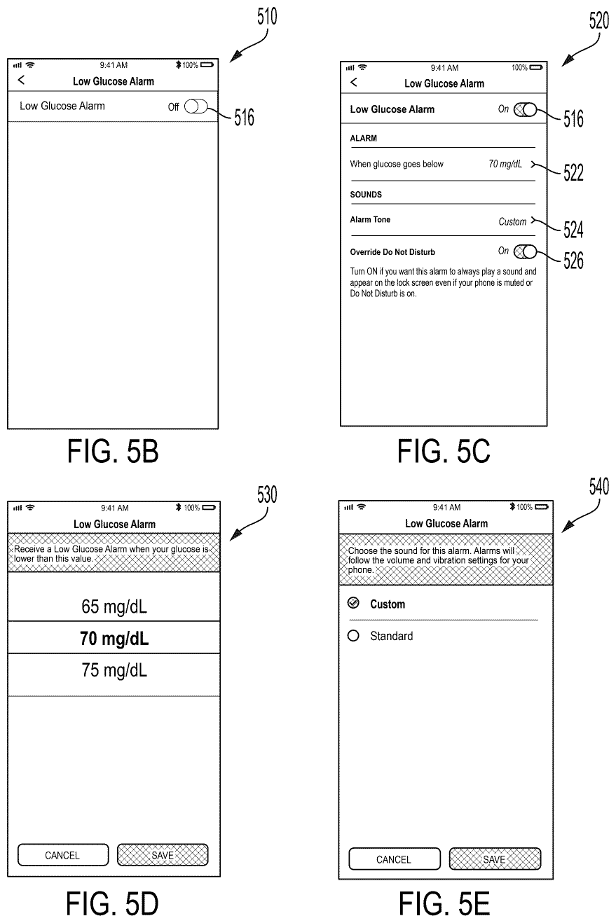

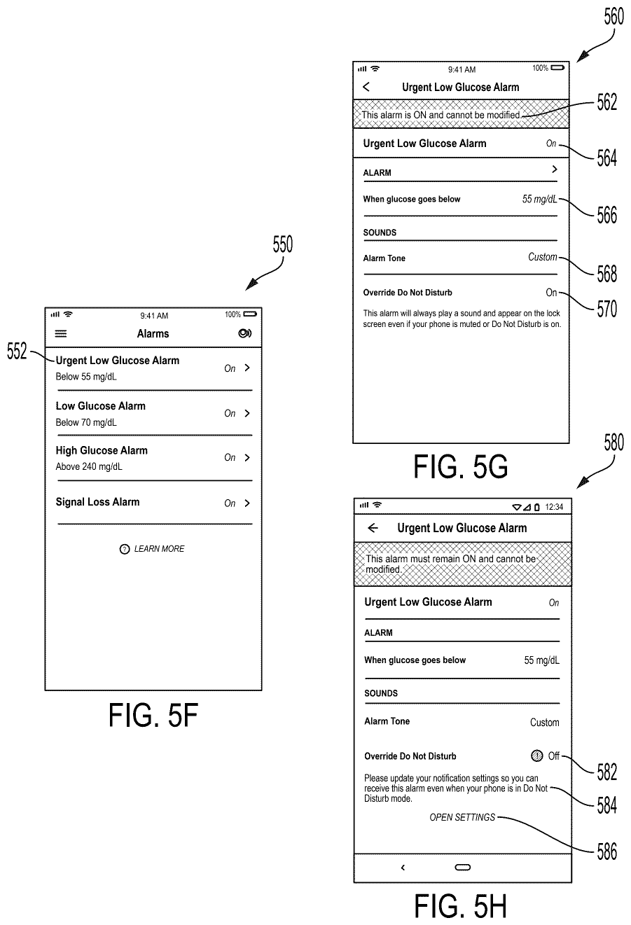

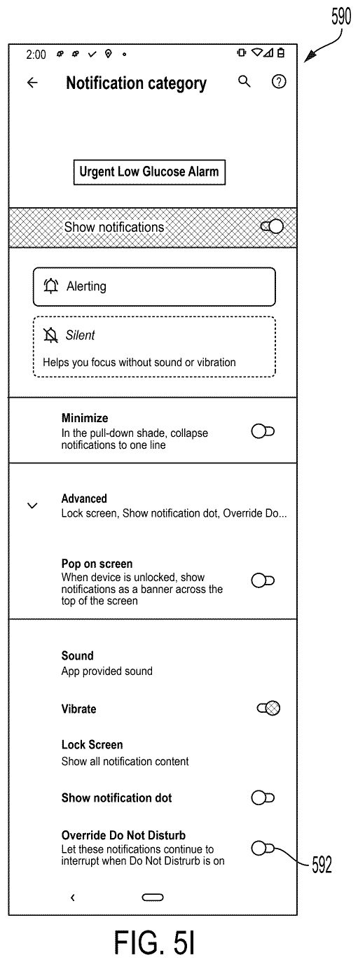

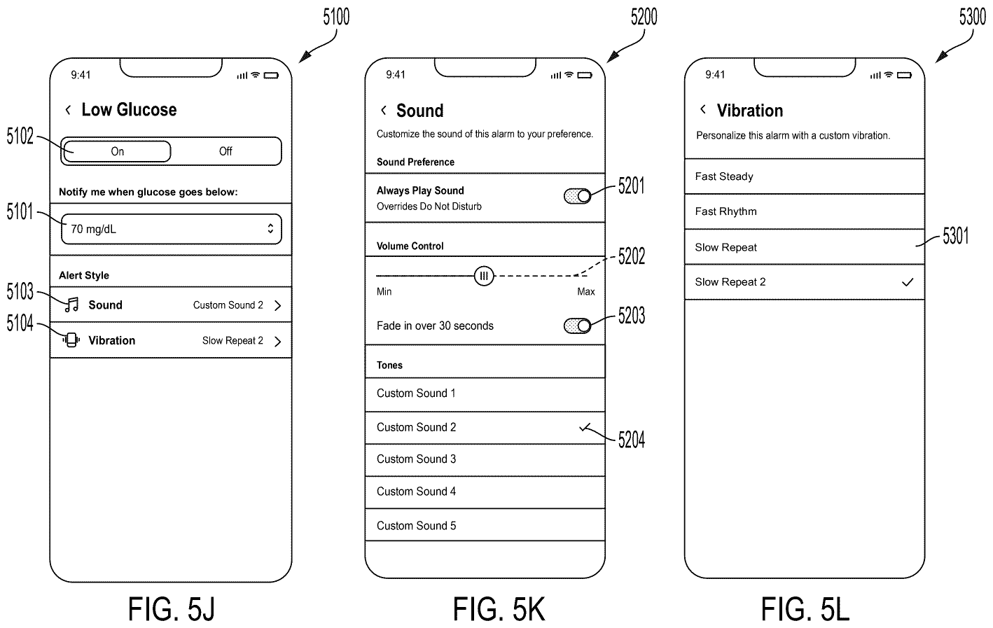

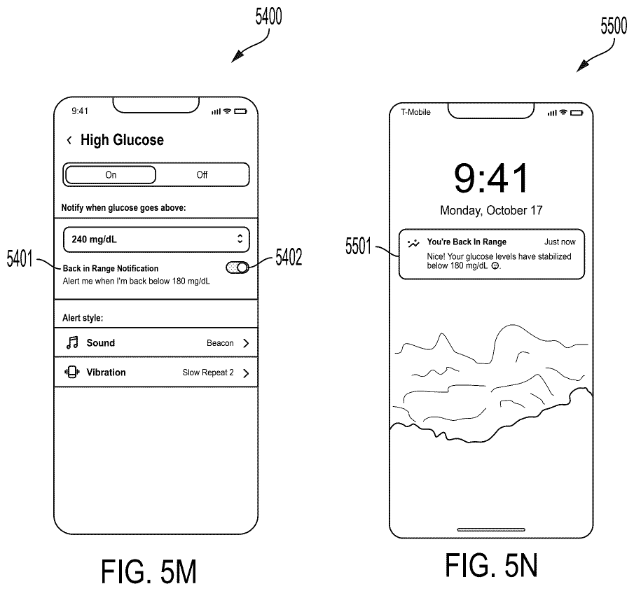

FIGS. 5A to 5E are example embodiments of GUIs including alarm settings for alarms in an analyte monitoring system. FIG. 5A is a GUI 500 depicting an Alarms settings interface comprising three selectable alarm options: a low glucose alarm option 502, a high glucose alarm option 504, and a signal loss alarm option 506. Adjacent to each selectable alarm option is a textual indicator to indicate whether the alarm is on or off. Additionally, in some embodiments, a selectable “Learn More” option for additional information is provided beneath the selectable alarms. FIG. 5B is a GUI 510 depicting a Low Glucose Alarms settings interface. According to one aspect of the embodiments, GUI 510 can be displayed when the user selects the Low Glucose Alarm in previous GUI 500. According to another aspect of the embodiments, GUI 510 includes a textual label for the “Low Glucose Alarm” adjacent to a switch 516 configured to be toggled between an on position and an off position. Those of skill in the art will understand that, instead of a toggle, GUI 510 can include any one or more of: an on-off checkbox, on-off slider switch, on-off radio buttons, on-off buttons, and the like. As seen in FIG. 5B, when switch 516 is in the off position, no other settings for the Low Glucose Alarm are available and/or visible.

FIG. 5C is a GUI 520 depicting the Low Glucose Alarms settings interface after the switch 516 has been toggled to the on position. After switch 516 has been toggled to the on position, as can be seen in FIG. 5C, GUI 520 can include a plurality of configurable settings, including (but not limited to) a low glucose alarm threshold setting 522, a low glucose alarm tone setting 524, and a low glucose alarm override setting 526. According to one aspect of the embodiments, the low glucose alarm threshold setting 522 can be configured by the user such that the user can select a low glucose threshold (as shown in GUI 530 of FIG. 5D), where a low glucose alarm will be triggered when the user's glucose level falls below the selected low glucose threshold. According to another aspect of the embodiments, the low glucose alarm tone setting 524 can be configurable by the user such that the user can select a standard alarm (e.g., adopting the operating system's alarm tone) or a custom low glucose alarm (e.g., allowing the user to select a specific tone or output), as shown in GUI 540 of FIG. 5E. According to some embodiments, the low glucose alarm tone can be either or both of an audible and a vibratory notification. According to another aspect of the embodiments, the low glucose alarm override setting 526 can be configured by the user such that, when enabled, a low glucose alarm will always output a sound (or vibration), and display a visual notification on the display of reader device 120 (e.g., on a lock screen), even if reader device 120 is muted or configured in a “Do Not Disturb” mode.

Although FIGS. 5B to 5E depict GUIs having configurable alarm settings for a low glucose alarm, those of skill in the art will recognize that similar GUIs can be utilized for configurable alarm settings for a high glucose alarm or a signal loss alarm, and are fully within the scope of the present disclosure.

FIGS. 5F to 5I are additional example embodiments of GUIs including alarm settings for alarms in an analyte monitoring system. FIG. 5F is a GUI 550 depicting an Alarms settings interface comprising four selectable alarm options: an urgent low glucose alarm option 552 (also referred to as a “urgent low glucose alarm” or a “fixed low glucose alarm”), a low glucose alarm option, a high glucose alarm option, and a signal loss alarm option. In one aspect, GUI 550 is similar to GUI 500 of FIG. 5A, except that GUI 550 further includes an urgent low glucose alarm 552. FIG. 5G is a GUI 560 depicting an Urgent Low Glucose Alarm settings interface. According to one aspect of the embodiments, GUI 560 can be displayed when the user selects the Urgent Low Glucose Alarm in previous GUI 550. According to another aspect of the embodiments, GUI 560 includes an informational section 562 that indicates that the Urgent Low Glucose Alarm “is ON and cannot be modified.” In addition, like GUI 520 of FIG. 5C, GUI 560 further includes: a textual label for “Urgent Low Glucose Alarm” adjacent to a switch 564, an urgent low glucose alarm threshold setting 566, an urgent low glucose alarm tone setting 568, and an urgent low glucose alarm override setting 570.

In many embodiments, the aforementioned settings are not configurable to the user, and displayed for informational purposes only. For example, unlike switch 516 in GUIs 510 and 520 (FIGS. 14B and 14C), switch 564 cannot be toggled to an “Off” position or state. Similarly, according to another aspect of some embodiments, the urgent low glucose alarm threshold setting 566, the urgent low glucose alarm tone setting 568, and the urgent low glucose alarm override setting 570 cannot be modified or disabled.

In other embodiments, one or more of the aforementioned settings may be configurable by the user, while the rest of the settings are displayed for information purposes only. For example, in certain embodiments, the textual label and switch 564, alarm threshold setting 566, and alarm override setting 570 can be non-configurable, while the alarm tone setting 568 may be configurable so as to allow the user to select a specific tone or vibration. Other combinations of configurable and non-configurable settings are possible, and those of skill in the art will recognize that these combinations are fully within the scope of the present disclosure.

According to another aspect of some embodiments, certain settings may become “active” under certain conditions. FIG. 5H is a GUI 580 depicting an Urgent Low Glucose Alarm settings interface, similar to GUI 560 of FIG. 5G. As seen at the bottom third of the interface, the urgent low glucose alarm override setting 582 is shown in an “Off” state. In addition, a critical alert icon or badge is displayed adjacent to the “Off” state, indicating that corrective action is needed. In some embodiments, further guidance 584 can be displayed on the interface (e.g., “Please update your notification settings so you can receive this alarm even when your phone is in Do Not Disturb Mode”). According to one aspect of some embodiments, GUI 580 presents an active “Open Settings” link 586 near the bottom of the interface. Upon selection of link 586, GUI 590 (FIG. 51) is displayed. In many of the embodiments, GUI 590 is a notification settings interface provided through the operating system of reader device 120, instead of from within the analyte monitoring software application running on reader device 120. According to another aspect of the embodiments, GUI 590 includes an “Override Do Not Disturb” setting 592, which can be enabled by the user. According to an aspect of some embodiments, once the override setting 592 is enabled, the “Open Settings” link 586 of GUI 580 can be transitioned to a hidden or inactive state.

Referring to FIGS. 5J to 5L, example embodiments of GUIs including alarm sound and vibration settings for alarms in an analyte monitoring system are depicted. Specifically, FIG. 5J is a GUI 5100 depicting an alarm settings interface. More specifically, in FIG. 5J, GUI 5100 depicts an exemplar embodiment of a Low Glucose Alarm sound settings interface. However, those of skill in the art will appreciate that the low glucose alarm features depicted in GUI 5100 are meant to be illustrative and non-exhaustive, and that GUI 5100 can be utilized for various other alarms (e.g., an urgent low glucose alarm, a high glucose alarm, and a signal loss alarm) without departing from the scope of the present disclosure.

According to one aspect of the embodiments, GUI 5100 can be displayed when the user selects the Low Glucose alarm in, e.g., GUI 550 (FIG. 5F). Similar to GUI 510 (FIG. 5C), GUI 5100 comprises a low glucose alarm threshold setting 5101. However, instead of a switch, as shown in FIG. 5C, GUI 5100 includes an on-off button 5102. Further, and unlike GUI 510 (FIG. 5C), GUI 5100 further comprises two selectable alert style settings: (1) a sound alert style setting 5103 and (2) a vibration alert style setting 5104. Further, each of the alert style settings 5103, and 5104 comprise a textual indicator 5104 which indicates to the user the selected style of the alert associated with the respective alert style settings 5103, 5104 (e.g., “Custom Sound 2” for the sound alert style setting 5103 and “Slow Repeat 2” for the vibration alert style setting 5104).

FIG. 5K depicts an alarm sound settings GUI 5200, which is outputted in response to the user selecting the sound alert style setting 5103 in GUI 5100 (FIG. 5J), and allows the user to customize the sound of the alarm associated with previous GUI 5100 (e.g., sound of the low glucose alarm). Specifically, in some embodiments, alarm sound settings GUI 5200 comprises a switch 5201 that can be toggled between an “on” state and an “off” state. Upon toggling switch 5201 to the “on” state, the user configures the alerts of the alarms associated with previous GUI to always play a sound. As such, alarm sounds will be outputted to override a “Do Not Disturb” feature of the underlying phone or device. Upon toggling the switch 5201 to the “off” state, the user turns off alerts corresponding to the alarms associated with previous GUI 5100. Further, in some embodiments, the alarm sound settings GUI 5200 further comprises a selectable volume control bar 5202. In some embodiments, the selectable volume control bar 5202 is configured to adjust the volume associated with the alert sounds. Specifically, and in response to a first predetermined input by the user, such as when the user drags the volume control bar 5202 with a finger, or by some other predetermined gesture, the user can adjust the volume between a range from a minimum volume level to a maximum volume level.

Further, in some embodiments, alarm sound settings GUI 5200 can also include a selectable volume fade option 5203 which, upon being selected, allows the user to fade the alarm to a personal maximum volume over a predetermined time (e.g., fades in over 30 seconds). In some embodiments, alarm sound settings GUI 5200 also comprises a plurality of selectable tone options 5204. In this regard, the user can select one of the plurality of selectable tone options 5204 so as to configure a custom discernable tone for the selected alarm.

FIG. 5L depicts an alarm vibration settings GUI 5300 which is outputted in response to the user selecting the vibration alert style setting 5104 in GUI 5100 (FIG. 5J), and allows the user to customize the vibration pattern of the alarm associated with previous GUI 5100 (e.g., sound of the low glucose alarm). Alarm vibration settings GUI 5300 comprises a plurality of selectable vibration pattern alert options 5301. In this regard, the user can select one of the plurality of selectable vibration pattern alert options 5301 so as to configure a custom vibration pattern for the selected alarm.

Those of skill in the art will appreciate that the aforementioned alarm sounds and vibration settings interfaces are merely illustrative and non-exhaustive, and that various other alarm sound and vibration settings interface associated with other alarms (e.g., an urgent low glucose alarm, a high glucose alarm, and a signal loss alarm) can be utilized without departing from the scope of the present disclosure.

Turning to FIGS. 5M to 5N, example embodiments of GUIs including “Back In Range” settings and features related thereto for alarms in an analyte monitoring system are depicted. Specifically, FIG. 5M is a GUI 5400 depicting an alarm settings interface. GUI 5400 is similar to GUI 5100 depicted in FIG. 5J, except that GUI 5400 depicts a High Glucose Alarm settings interface. Additionally, GUI 5400 includes a “Back In Range” notification option 5401 with a switch 5402 that can be toggled between an “on” state and an “off” state. Upon toggling switch 5402 to the “on” state, the user configures the alerts of the “Back In Range” notifications associated with the alarm to be turned on. Upon toggling the switch 5402 to the “off” state, the user turns off alerts of the “Back In Range” notifications associated with the alarm. In exemplar embodiments, when the “Back In Range” notification option 5401 is in the “on” state, the user will be alerted when the user has returned to a predetermined analyte range (e.g., below a predetermined analyte threshold level, such as, below 180 mg/dL). Though GUI 5400 illustrates the alarm settings interface for a High Glucose Alarm, those of skill in the art will appreciate that GUI 5400 can be utilized for various other alarm options, such as an urgent low glucose alarm, low glucose alarm, signal loss alarm, and the like, without departing from the scope of the disclosure. As such, the user can turn on notifications related to “Back In Range” for various alarms of the analyte monitoring software application.

Referring next to FIG. 5N, an example embodiment of a lock screen GUI 5500 is depicted, wherein a “Back In Range” notification 5501 is displayed to the user to indicate that the user had returned to the predetermined analyte range (e.g., below a predetermined analyte threshold level, such as, below 180 mg/dL). In some embodiments, the “Back In Range” notification 5501 can present a motivating message to the user and/or notify the user of their specific analyte level (e.g., “Nice! Your glucose levels have stabilized below 180 mg/dL).

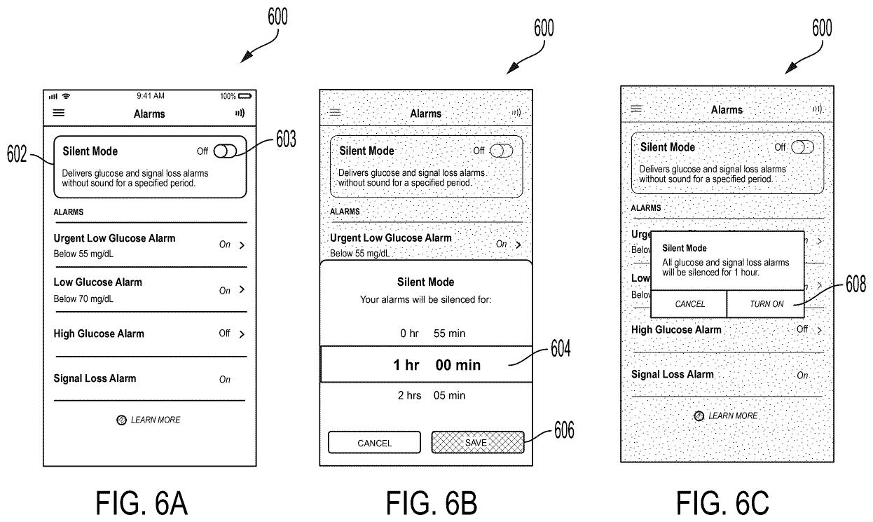

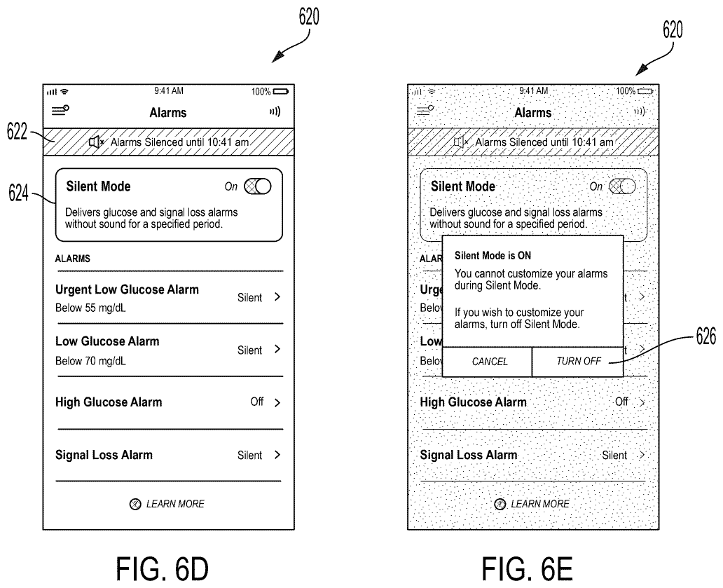

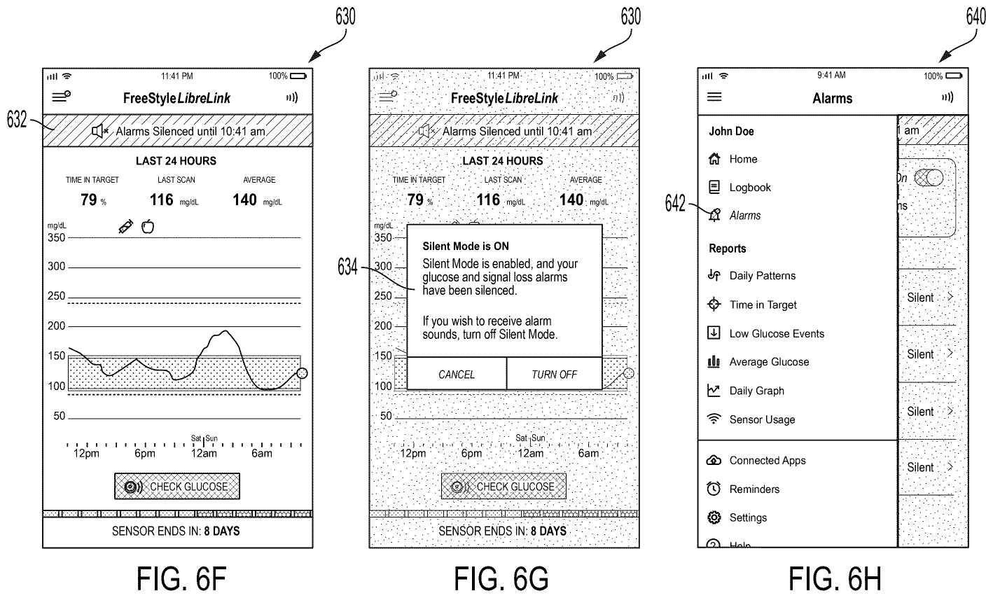

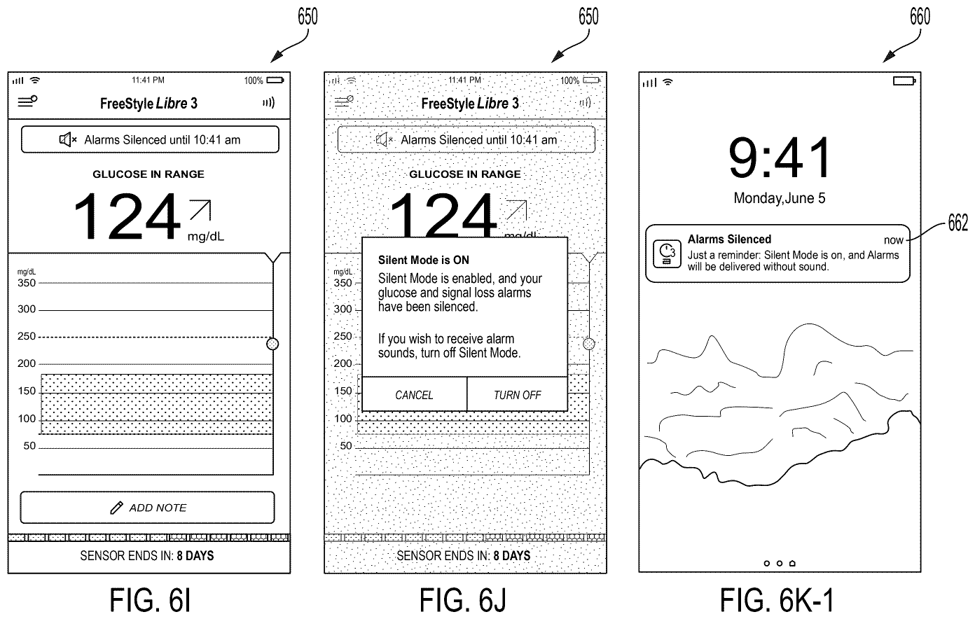

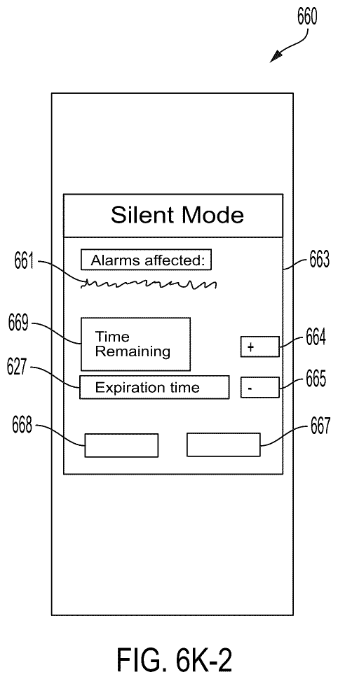

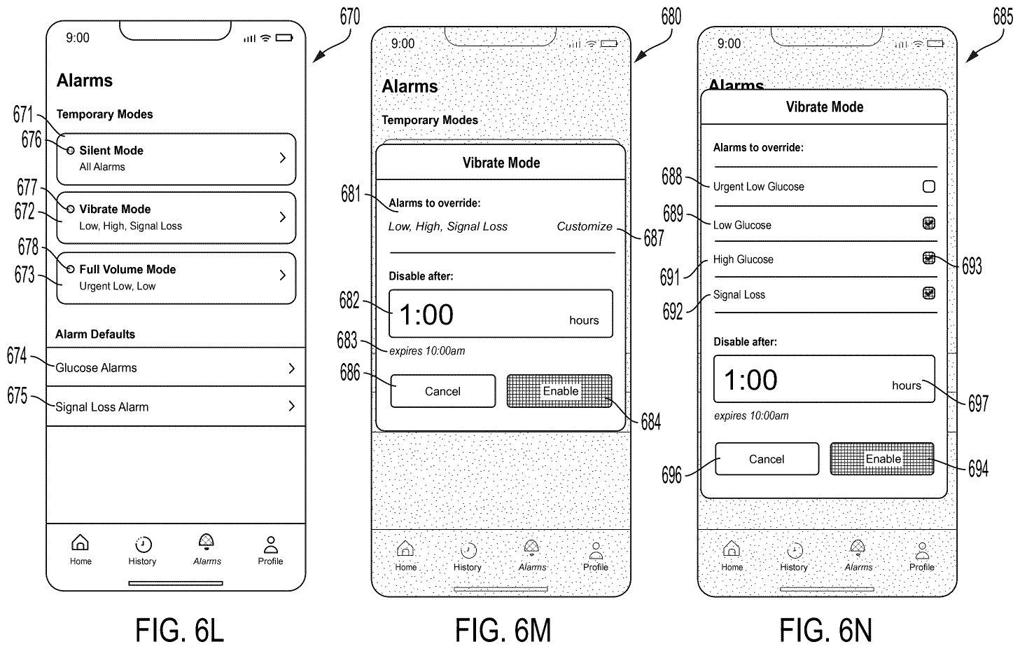

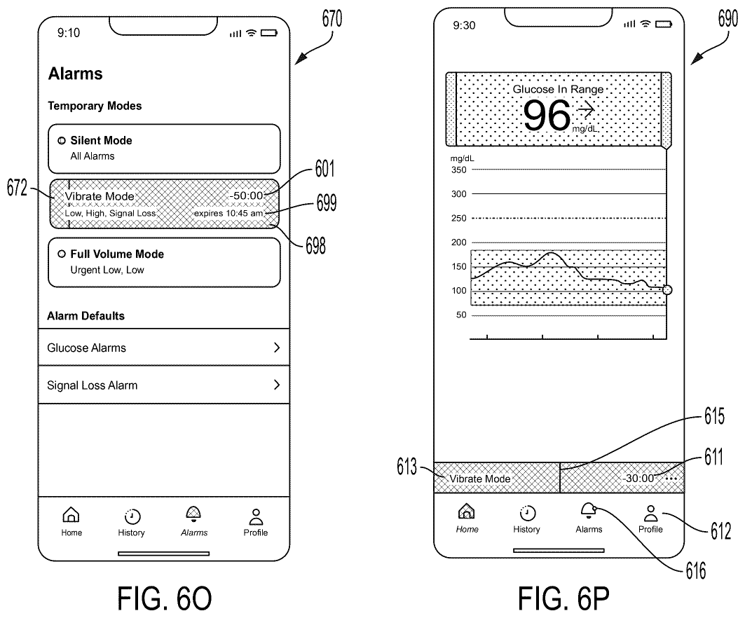

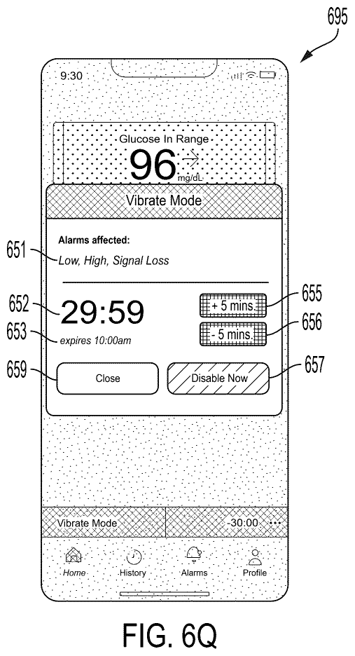

FIGS. 6A to 6K-2 are additional example embodiments of GUIs including alarm settings for alarms in an analyte monitoring system, with respect to a Silent Mode feature. In some instances, a user of an analyte monitoring system may be in an environment in which the user desires to temporarily silence alarms (e.g., a movie theater, business meeting, quiet area, etc.). Several example embodiments described herein include a Silent Mode feature of an analyte monitoring software application that can be enabled for a predetermined period of time in order to temporarily prevent alarms from outputting an audible noise. In some embodiments, the alarms can continue to output vibratory alerts. In other embodiments, enabling Silent Mode can prevent any type of alarm output.

Referring to FIGS. 6A to 6C, an example embodiment of an alarm setting GUI 600 for an analyte monitoring software application is depicted, wherein the alarm setting GUI 600 includes a Silent Mode feature 602. According to one aspect of the embodiment, alarm setting GUI 600 resembles the alarm setting GUIs of FIGS. 5A and 5F, except that alarm setting GUI 600 also includes a Silent Mode feature 602, shown here at the top of the interface. In many embodiments, the Silent Mode feature 602 includes a switch 603 that can be toggled between an “on” state and an “off” state. Upon toggling switch 603 to the “on” state, a configuration modal 604, as seen in FIG. 6B, is displayed to the user. The user can specify the amount of time for Silent Mode to be enabled. In some embodiments, configuration modal 604 can have a maximum time limit such that the user cannot enable Silent Mode for longer than such (e.g., a maximum time limit of six hours). In some embodiments, configuration modal 604 can have a minimum time limit such that the user cannot enable Silent Mode for shorter than such (e.g., a minimum time limit of five minutes). In some embodiments, configuration modal 604 can include a “Save” button 606, which will save the user's selection in terms of the configurable duration of Silent Mode. Upon actuating “Save” button 606, the user can be prompted with another confirmation modal 608, as shown in FIG. 6C, which indicates to the user that “all glucose and signal loss alarms will be silenced” for the selected amount of time. In many embodiments, the confirmation modal 608 can include a “Turn On” option to proceed with enabling Silent Mode, and/or a “Cancel” option, in case the user does not want to proceed with Silent Mode.

Although not depicted, in some embodiments, alarm settings GUI 600 can also prompt the user for authentication before enabling Silent Mode. In some embodiments, for example, the authentication step can comprise prompting the user to enter a password or PIN number. In some embodiments, the authentication step can comprise a biometric routine, such as a facial recognition routine performed by the operating system of the reader device. In some embodiments, the authentication step can be performed in response to the toggling of switch 603 from an “off” state to an “on” state. In some embodiments, the authentication step can be performed in response to the user actuating the “Save” button 606 or “Turn On” button 608, as shown in FIGS. 6B and 6C.

Referring next to FIGS. 6D and 6E, another example embodiment of an alarm settings GUI 620 for an analyte monitoring software application is depicted, wherein the alarm settings GUI 620 also includes an enabled Silent Mode feature 624. According to one aspect of the embodiment, alarm settings GUI 620 resembles the alarm settings GUI 600 of FIG. 6A. In some embodiments, alarm settings GUI 620 includes an in-app banner notification 622 to indicate to the user that Silent Mode is enabled until an expiration time (e.g., “Alarms Silenced until 10:41 am”). In other embodiments, the in-app banner notification 622 can indicate to the user how much time is remaining until Silent Mode expires (e.g., “Alarms Silenced for 2 hours and 30 minutes”).

Referring still to FIGS. 6D and 6E, according to another aspect of some embodiments, if the user attempts to configure the alarms (e.g., by selecting “Low Glucose Alarm”), modal 626 can be displayed to the user to indicate that alarms cannot be customized during Silent Mode, as shown in FIG. 6E. In some embodiments, modal 626 can include a selectable “Turn Off” button, to allow the user to immediately disable Silent Mode.

Turning now to FIGS. 6F and 6G, an example embodiment of a home screen GUI 630 for an analyte monitoring software application is depicted, wherein a Silent Mode feature has been enabled. According to one aspect of many embodiments, home screen 620 can include an in-app banner notification 632 to indicate to the user that Silent Mode is enabled until an expiration time (e.g., “Alarms Silenced until 10:41 am”). In other embodiments, the in-app banner notification 622 can indicate to the user how much time is remaining until Silent Mode expires (e.g., “Alarms Silenced for 2 hours and 30 minutes”). In some embodiments, in-app banner notification 632 can also be selectable and configured to display, upon selection by the user, a modal 634 to provide further information to the user regarding Silent Mode, as shown in FIG. 6G. In some embodiments, modal 634 can also include a “Turn Off” button, to allow the user to immediately disable Silent Mode. Similarly, another example embodiment of a sensor results GUI 650 with an in-app banner notification to indicate that Silent mode is enabled is depicted in FIGS. 6I and 6J.

Referring next to FIG. 6H, an example embodiment of a navigation menu GUI 640 is depicted, wherein navigation menu GUI 640 includes an indicator 642 next to the selectable Alarms option. In many embodiments, indicator 642 conveys to the user that Silent Mode is currently enabled.

Referring next to FIG. 6K-1, an example embodiment of a lock screen GUI 660 is depicted, wherein a Silent Mode notification 662 is displayed to the user to indicate that Silent Mode is enabled. In some embodiments, Silent Mode notification 662 indicates to the user that Silent Mode is enabled until an expiration time (e.g., “Alarms Silenced until 10:41 am” or “Silent Mode is on until 10:41 am”). In other embodiments, Silent Mode notification 662 can indicate to the user how much time is remaining until Silent Mode expires (e.g., “Alarms Silenced for 2 hours and 30 minutes”).

Still referring to FIG. 6K-1, in some embodiments, the Silent Mode notification 662 can be periodically outputted to the lock screen GUI 660 throughout the duration of the Silent Mode period (e.g., every 30 minutes, every hour, every two hours, etc.). In other embodiments, the Silent Mode notification 662 can be outputted to the lock screen GUI 660 in response to one or more predetermined events (e.g., user closing or placing the analyte monitoring software application in the background). In some embodiments, the Silent Mode notification 662 can be outputted to the lock screen GUI 660 after a predetermined period of time has elapsed before the expiration time. For example, in some embodiments, the Silent Mode notification is outputted after half or at least half the duration of expiration time has elapsed, or when half or at least half the expiration time of Silent Mode is remaining (e.g., if the alarms are silenced for one hour, then the Silent Mode notification 662 is outputted 30 minutes after Silence Mode is turned on). Those of skill in the art will recognize that the Silent mode notification 662 can be outputted after various other durations of time have elapsed prior to the expiration time (e.g., when 25% of the time has elapsed prior to expiration time, when 75% of the time has elapsed prior to expiration time), without departing from the scope of the present disclosure.

According to an aspect of the embodiments, and with reference to FIG. 6K-1, the Silent Mode notification 662 can be outputted silently to the lock screen GUI 660. In some embodiments, the Silent Mode notification 662 is outputted silently to the lock screen GUI 660 even when all other device notifications external to the analyte monitoring software application present auditory outputs. In some embodiments, the Silent Mode notification 662 can present a vibratory output when outputted to the lock screen GUI 660.

In some embodiments, and as shown in FIG. 6K-2, Silent Mode notification 662 can be expanded into a Silent Mode modal 663 in response to a second predetermined input by the user, such as when the user taps, drags, long-presses for a predetermined press time, swipes with a finger, or by some other predetermined gesture on the Silent Mode notification 662 illustrated in FIG. 6K-1. In some embodiments, and as illustrated in FIG. 6K-2, the Silent Mode modal 663 is outputted to lock screen GUI 660 and provides the user information related to the Silent Mode settings. For example, in some embodiments, the Silent Mode modal 663 can comprise one or more of the following: (1) a list 661 indicating to the user which alarms are affected by Silent Mode (e.g., all alarms); (2) a countdown timer 669 indicating the duration of time remaining until Silent Mode expires (e.g., “29:59” to indicate 29 minutes and 59 seconds are remaining until Silent Mode expires); and (3) a textual indicator 627 which indicates the expiration time of the Silent Mode (e.g., “10:00 am”). Further, in some embodiments, the user can modify Silent Mode settings through the Silent Mode modal 663. Specifically, in some embodiments, the Silent Mode modal 663 comprises an “add time” button 664 which, upon being selected by the user, allows the user to increase the duration of Silent Mode. In some embodiments, the add time button 664 allows the user to increase the duration of Silent Mode by a predetermined increment of time. For example, each time the user selects the add time button 664, a predetermined amount of time is added to the Silent Mode expiration time (e.g., five minutes is added to the Silent Mode expiration time). Further, in some embodiments, the Silent Mode modal further comprises a “reduce time” button 665 which, upon being selected by the user, allows the user to reduce the duration of Silent Mode. In some embodiments, the reduce time button 665 allows the user to decrease the duration of Silent Mode by a predetermined increment of time. For example, each time the user selects the reduce time button 654, a predetermined amount of time is reduced from the Silent Mode expiration time (e.g., five minutes is reduced from the Silent Mode expiration time). Further, in some embodiments, the user can immediately disable Silent Mode through the Silent Mode modal 663 by selecting a “Turn Off” button or “Disable Now” button 667. To close or exit the Silent Mode modal 663, the user can select a “Close” button 668 provided on the Silent Mode modal 663.

FIGS. 6L to 6Q are additional example embodiments of GUIs including alarm settings for alarms in an analyte monitoring system, with respect to a Temporary Mode feature. In some instances, a user of an analyte monitoring system may be in an environment in which the user desires to temporarily silence alarms or enable vibratory alerts for alarms (e.g., a movie theater, business meeting, quiet area, etc.), or enable a maximum volume for an alarm (e.g., for a low glucose condition or a serious low glucose condition). Several example embodiments described herein include a Temporary Mode feature of an analyte monitoring software application which allows the user to select from various different Temporary Modes to enable for a predetermined period of time in order to temporarily prevent alarms from outputting an audible noise and/or temporarily enable the output of maximum volume alerts for alarms. In some embodiments, the maximum volume would be the volume to which the reader device's volume is currently set. In other embodiments, the maximum volume can be a preset or default volume set by the analyte monitoring software application.

According to one aspect of the embodiments, and with reference to FIG. 6L, another example embodiment of an alarm settings GUI 670 for an analyte monitoring software application is depicted, wherein the alarm settings GUI 670 comprises three selectable Temporary Mode alarm options: a Silent Mode option 671 which upon being selected can temporarily prevent any or selected types of alarm output, a Vibrate Mode option 672 which upon being selected can temporarily enable vibratory alerts for any or selected types of alarm output, and a Full Volume Mode option 673 which upon being selected can temporarily enable maximum volume alerts any or selected types of alarm output. In some embodiments, the user can customize the name of each selectable Temporary Mode alarm option. Each selectable Temporary Mode alarm option includes a textual indicator to indicate which alarms are overridden by each respective Temporary Mode alarm option (as shown in FIG. 6L, for example, Silent Mode option 671 includes a textual indicator indicating that it is configured to override all alarms, Vibrate Mode option 672 includes a textual indicator indicating that it is configured to override the low glucose alarm, high glucose alarm, and signal loss alarm, and Full Volume Mode option 673 includes a textual indicator indicating that it is configured to override the urgent low glucose alarm and the low glucose alarm). According to an aspect of the embodiments, once a Temporary Mode is activated, it must be disabled or expired before another Temporary Mode can be selected by the user. In other embodiments, the user can contemporaneously enable one or more Temporary Modes. In some embodiments, each selectable Temporary Mode alarm option further includes a colored dot 676, 677, 678, wherein the colored dot 676, 677, 678 of each selectable Temporary Mode alarm option is different and indicative of its respective Temporary Mode alarm option. For example, as shown in FIG. 60, a first colored dot 676 (e.g., a purple colored dot) is associated with the Silent Mode option 671, a second colored dot 677 (e.g., a navy colored dot) is associated with the Vibrate Mode option 672, and a third colored dot 678 (e.g., a blue colored dot) is associated with the Full Volume Mode option 673. Those of skill in the art will appreciate that other colors, markers, and indicators can be utilized to differentiate each Temporary Mode alarm option without departing from the scope of the present disclosure.

Still referring to FIG. 60, in some embodiments, alarm settings GUI 670 further comprises two selectable alarm default options: a glucose alarm default option 674 and a signal loss alarm default option 675. Specifically, and upon the user selecting an alarm default option, an interface is outputted to the user which indicates the default settings for the alarms associated with the selected alarm default option.

Referring back to FIG. 6M, upon the user selecting a Temporary Mode alarm option, a confirmation screen 680 associated with the selected Temporary Mode alarm option is outputted, wherein the confirmation screen 680 can comprise (1) an alarm override indicator 681 which lists the alarms affected by the selected Temporary Mode alarm option is configured to override, and (2) an expiration time configurator 682 which allows the user to set the duration for the selected Temporary Mode alarm option associated with the confirmation screen 680. For example, as shown in FIG. 6M, a confirmation screen 680 for the Vibrate Mode option 672 is displayed in response to the user selecting the Vibrate Mode option 672 in alarm settings GUI 670 (FIG. 6L), wherein the alarm override indicator 681 indicates the low glucose alarm, high glucose alarm, and signal loss alarm are overridden by the Vibrate Mode option 672. Further expiration time configurator 682 illustrated in FIG. 6M indicates that the expiration time for Vibrate Mode option 672 is set for one hour. The confirmation screen 680 can further comprise a textual indicator 683 which informs the user of the expiration time associated with the selected Temporary Mode alarm option (as shown in FIG. 6M, for example, the textual indicator 683 indicates an expiration time of 10:00 AM). In some embodiments, confirmation screen 680 can include an “Enable” button 684, which will enable the Temporary Mode alarm option corresponding to the outputted confirmation screen 680. In some embodiments the confirmation screen 680 further includes a “Cancel” button 686, in case the user does not want to proceed with the Temporary Mode alarm option associated with the outputted confirmation screen 680.

Still referring to FIG. 6M, and according to some embodiments, adjacent to the alarm override indicator 681 on the confirmation screen 680 is a selectable “Customize” option which, upon being selected by the user, outputs a configurator modal 685 (FIG. 6N) which allows the user to adjust the settings associated with the selected Temporary Alarm option corresponding to the confirmation screen 680. Specifically, the user can customize the alarms affected by the selected Temporary Mode alarm option through the configurator modal 685. As depicted in FIG. 6N, the configurator modal 685 comprises a list of selectable alarm override options which allows the user to select the alarms the Temporary Mode alarm option can be configured to override. In some embodiments, the list of selectable alarm override options can comprise: (1) a selectable urgent low glucose alarm override option 688, (2) a selectable low glucose alarm override option 689, (3) a selectable high glucose alarm override option 691, and (4) a selectable signal loss alarm override option 692. Adjacent to each selectable alarm override option is a checkbox 693 configured to toggle between an on position (checked configuration) and an off position (unchecked configuration). In this manner, if the user wants a particular alarm to be overridden by the selected Temporary Mode alarm option, the user can toggle the checkbox 693 to the on position or checked configuration. Further, if the user wants a particular alarm to not be overridden by the selected Temporary Mode alarm option, the user can toggle the checkbox 693 to the off position or unchecked configuration. For example, in the exemplary embodiment of the configurator modal 685 shown in FIG. 6N, the Vibrate Mode option 672 is configured to override the low glucose alarm, the high glucose alarm, and the signal loss alarm, but not the urgent low glucose alarm. Those of skill in the art will understand that, instead of a checkbox, each selectable alarm override option shown on configurator modal 685 can include any one or more of: an on-off toggle, on-off slider switch, on-off radio buttons, on-off buttons, and the like.

Still referring to FIG. 6N, and in some embodiments, configurator modal can include an expiration time configurator 697 which allows the user to adjust the duration for a particular Temporary Mode alarm option. Further, configurator modal 685 can include an “Enable” button 694, which will enable the user to save the settings corresponding to the selected Temporary Mode alarm option. In some embodiments the configurator modal 685 further includes a “Cancel” button 696, in case the user does not want to proceed with adjusting the settings associated with the selected Temporary Mode alarm option. According to an aspect of the embodiments, any adjustments made to settings and saved through the configurator modal 685 are automatically saved for the user's subsequent activation of the Temporary Mode alarm option associated therewith.

With reference again to FIG. 60, alarm settings GUI 670 is illustrated, wherein one of the three selectable Temporary Mode alarm options is in an active or enabled state. Specifically, when a Temporary Mode alarm option is active or enabled, the active Temporary Mode alarm option comprises a colored banner 698 to indicate its active or enabled state. For example, as shown in FIG. 60, the Vibrate Mode alarm option 672 comprises a colored banner 698 (e.g., a navy banner) which corresponds to the color of the colored dot 676, 677, or 678 (FIG. 6L) associated with the enabled Temporary Mode alarm option. Specifically, in FIG. 60, the Vibrate Mode alarm option 672 comprises a navy colored banner 698 to indicate that the Vibrate Mode alarm option 672 is in an enabled state. Further, according to an aspect of some embodiments, when one of the selectable Temporary Mode alarm options is enabled, the enabled Temporary Mode alarm option includes a textual indicator 699 indicating the expiration time of the enabled temporary Mode alarm option (e.g., “expires 10:45 am”). In some embodiments, the enabled Temporary Mode alarm option also includes a countdown timer 601 indicating the time remaining until the expiration time (e.g., −50:00). In some embodiments, the countdown timer 601 can be configured to indicate the time elapsed since activation of the Temporary Mode alarm option. In this manner, the temporary, but still urgent, nature of the enabled mode is emphasized to the user.

FIG. 6P is an exemplar embodiment of a GUI 690 depicting an analyte graph, wherein the GUI 690 comprises a Temporary Mode status bar 611 which is displayed near, on, or directly adjacent to the navigation menu 612 of GUI 690. It will be understood by those of skill in the art that the GUI embodiments displaying the Temporary Mode status bar 611 or banner 698 described herein, are meant to be illustrative only, and that Temporary Mode status bar 611 or banner 698 611 can be displayed on any interface of the analyte monitoring software application without departing from the scope of the present disclosure. According to an aspect of the embodiments, Temporary Mode status bar 611 is fixed to the navigation menu 612 of a displayed interface until the expiration of the enabled alarm associated therewith. In some embodiments, the navigation menu 612 includes an indicator 616 next to a selectable alarm option to convey to the user that a Temporary Mode is currently enabled. Specifically, according to an aspect of the embodiments, the indicator 616 comprises a color which corresponds to the colored dot 676, 677, or 678 (FIG. 6L) associated with the enabled Temporary Mode alarm option. In FIG. 6P, for example, a navy indicator 616 is displayed next to the selectable alarm option to convey to the user that the Vibrate Mode alarm option 672 (depicted in FIG. 6L) is active.

In some embodiments, Temporary Mode status bar 611 comprises a textual indicator 613 which indicates the Temporary Mode alarm option that is in the enabled or active state (e.g., “Vibrate Mode,” as shown in FIG. 6P). Further, in some embodiments, and as depicted in FIG. 6P, Temporary Mode status bar 611 can further comprise a countdown timer 614 indicating the time remaining until the expiration time (e.g., “−30:00,” as shown in FIG. 6P, to indicate that 30 minutes remains until the enabled Temporary Mode expires). According to another aspect of the embodiments, the Temporary Mode status bar 611 further comprises a colored portion 615, wherein the colored portion matches with or corresponds to the colored dot 676, 677, or 678 associated with the enabled Temporary Mode alarm option. For example, and as depicted in FIG. 6P, the colored portion 615 comprises a navy colored portion 615, which corresponds to the navy colored dot 677 associated with the Vibrate Mode alarm option 672 depicted in FIG. 6L. In this regard, the color mapping allows the user to quickly discern which Temporary Mode option has been enabled.

According to another aspect of the embodiments, and with reference to FIG. 6Q, when the user taps or otherwise selects the Temporary Mode status bar 611 (FIG. 6P) associated with an active Temporary Mode alarm option, or on the banner 698 (FIG. 60) associated with active Temporary Mode alarm option, a Temporary Mode modal 695 is outputted (FIG. 6Q). In some embodiments, and as shown in FIG. 6Q, Temporary Mode modal 695 is configured to overlay the underlying interface of the analyte monitoring software application. Temporary Mode modal 695 allows the user to review the overridden alarms associated with the active Temporary Mode alarm option, adjust the remaining time, and/or immediately disable the Temporary Mode. Specifically, in some embodiments, Temporary Mode modal 695 can comprise one or more of the following: (1) a list 651 indicating to the user which alarms are affected or overridden by the active Temporary Mode alarm option (e.g., “Low, High, Signal Loss”); (2) a countdown timer 652 informing the user the duration of time remaining until the enabled Temporary Mode expires (e.g., “29:59” to indicate that 29 minutes and 59 seconds remain before expiration of the Vibrate Mode alarm option, as shown in FIG. 6Q); and (3) a textual indicator which indicates to the user the expiration time of the enabled Temporary mode alarm option (e.g., “expires 10:00 am”)

Further, in some embodiments, the user can modify the Temporary Mode settings associated with the enabled Temporary Mode alarm option through Temporary Mode modal 695. Specifically, in some embodiments, the Temporary Mode modal 695 further comprises an “add time” button 655 which, upon being selected by the user, allows the user to increase the duration of the enabled Temporary Mode. In some embodiments, the add time button 655 allows the user to increase the duration of the enabled Temporary Mode by a predetermined increment of time. For example, each time the user selects the add time button 655, a predetermined amount of time is added to the enabled Temporary Mode expiration time (e.g., five minutes is added to the Temporary Mode expiration time). Further, in some embodiments, the Temporary Mode modal 695 further comprises a “reduce time” button 656 which, upon being selected by the user, allows the user to reduce the duration of the enabled Temporary Mode. In some embodiments, the reduce time button 656 allows the user to decrease the duration of the enabled Temporary Mode by a predetermined increment of time. For example, each time the user selects the reduce time button 656, a predetermined amount of time is reduced from the enabled Temporary Mode expiration time (e.g., five minutes is reduced from the Temporary Mode expiration time). Further, in some embodiments, the user can immediately disable the active Temporary Mode through the Temporary Mode modal 695 by selecting a “Disable Now” button 657. To close or exit the Temporary Mode modal 695, the user can select a “Close” button 658 provided on the Temporary Mode modal 695.

FIG. 6Q, for example, depicts a Temporary Mode modal 695 associated with the Vibrate Mode alarm option. Those of skill in the art will appreciate that Temporary Mode modals can be outputted which are associated with Silent Mode (e.g., FIG. 6K-2) or Full Volume Mode (not illustrated).

It will be understood by those of skill in the art that any of the Temporary Mode GUIs (or portions thereof) described herein (e.g., FIGS. 6L to 6Q), are meant to be illustrative only, and that the individual elements, or any combination of elements, depicted and/or described for a particular embodiment or figure are freely combinable with any other element, or any combination of other elements, depicted and/or described with respect to any of the embodiments of the Silent Mode GUIs (or portions thereof) (e.g., FIGS. 6A to 6K-2).

According to another aspect of some embodiments, the analyte monitoring software application can be configured to provide escalating alarms. In particular, during an initial alarm presentation, the analyte monitoring software application can present a vibratory output. Then, for each subsequent alarm presentation, the analyte monitoring software application can present an auditory output at an increased volume. According to some embodiments, the subsequent alarm presentations can continue to output at an increased volume at a predetermined interval until reaching a maximum volume. In some embodiments, for example, the volume of the alarm can be increased every 10 seconds, every 30 seconds, every minute, etc.

Additionally, in some embodiments, the alarm settings GUI can include an interface such that the user can enable, disable, and configure escalating alarms. For example, in some embodiments, the user can select the interval at which the volume of the escalating alarm increases. In other embodiments, the user can select a specific sound (or sound file) to utilize with the escalating alarm mode. In still other embodiments, the escalating alarm setting can be an optional parameter associated with each of the alarms (e.g., Low Glucose Alarm, High Glucose Alarm, Signal Loss Alarm). In this regard, the user can utilize escalating alarms only for specific alarm conditions. In still other embodiments, the maximum volume of an escalating alarm can depend on whether the analyte monitoring software application has an Override Do-No-Disturb (“Override DND”) setting enabled. In some embodiments, for example, if the Override DND setting is enabled, then the maximum volume would be the volume to which the reader device's volume is currently set. On the other hand, in some embodiments, if the Override DND setting is disabled, the maximum volume can be a preset or default volume set by the analyte monitoring software application.

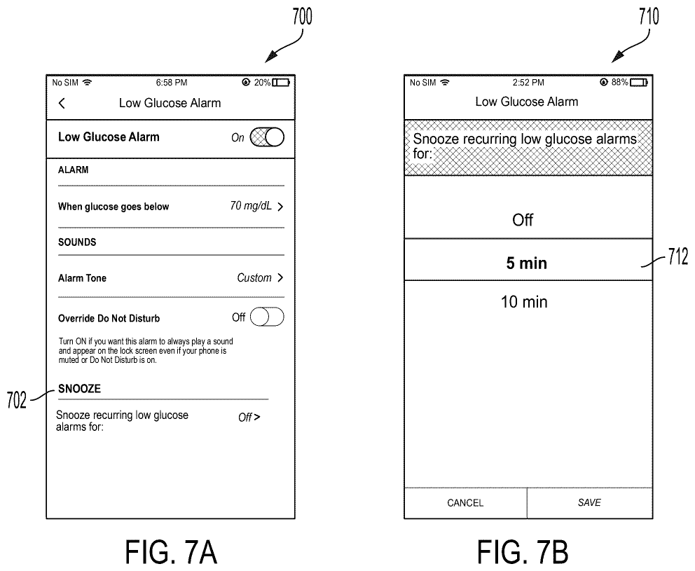

According to another aspect of some embodiments, the analyte monitoring software application can be configured to provide a snooze feature. In particular, during an alarm presentation, a prompt can be displayed to the user with a “snooze” option that allows the user to immediately stop the alarm output from continuing (e.g., “clear” the alarm). However, according to an aspect of some embodiments, selection of the “snooze” option does not dismiss the alarm. Accordingly, if the alarm condition persists, the alarm can present itself within five (5) minutes. In some embodiments, the user can configure the snooze feature to indicate the amount of time to prevent the alarm from generating an output (without dismissing the alarm). In still other embodiments, the snooze setting can be an optional parameter associated with each of the alarms of the analyte monitoring software application (e.g., Low Glucose Alarm), such as shown in GUIs 700 and 710 of FIGS. 7A and 7B. In this regard, the user can utilize the snooze feature only for specific alarm conditions.

Although the above descriptions of the figures and embodiments refer to alarms and alarm interfaces for a reader device, those of skill in the art will appreciate that these alarms and alarm interfaces can also be implemented in a sensor control device, a local computing system, a trusted computing system, or any other computing device within, or in communication with, an analyte monitoring system. Moreover, as described earlier, any of the GUIs and features described herein can comprise instructions stored in memory of a reader device, sensor control device, or any other computing device that is part of, or in communication with, the analyte monitoring system.

### Example Embodiments of Interfaces for Caregiver Applications and Alarms

Example embodiments of methods and interfaces relating to caregiver applications and alarms will now be described. As an initial matter, it will be understood by those of skill in the art that the GUIs and related methods described herein comprise instructions stored in a memory of reader device 120, local computer system 170, trusted computer system 180, and/or any other device or system that is part of, or in communication with, analyte monitoring system 100. These instructions, when executed by one or more processors of the reader device 120, local computer system 170, trusted computer system 180, or other device or system of analyte monitoring system 100, cause the one or more processors to perform the method steps and/or output the GUIs described herein. Those of skill in the art will further recognize that the GUIs described herein can be stored as instructions in the memory of a single centralized device or, in the alternative, can be distributed across multiple discrete devices in geographically dispersed locations.

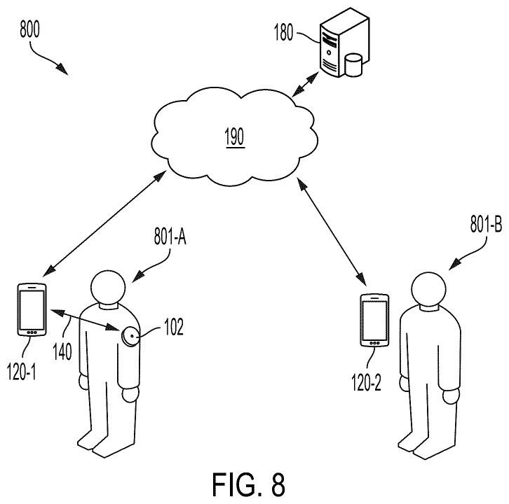

FIG. 8 is a conceptual diagram depicting an example embodiment of an analyte monitoring system 800 capable of communicating analyte data and other information to a caregiver. According to one aspect of the embodiments, analyte monitoring system 800 is generally similar to analyte monitoring system 100, as described with respect to FIG. 1. In particular, analyte monitoring system 800 can comprise sensor control device 102 (worn by patient 801-A) in wireless communication with reader device 120-1 via communication link 140. According to many of the embodiments, sensor control device 102 can include communication circuitry configured to wirelessly communicate data with reader device 120-1 via a Bluetooth communication protocol or Near Field Communication protocol. According to some embodiments, reader device 120-1 can be a smart phone, receiver device, or glucose meter. Furthermore, reader device 120-1 can be in wireless communication with trusted computer system 180 through network 190. In some embodiments, reader device 120-1 can include communication circuitry configured to wirelessly communicate data with trusted computer system 180 via an 802.11x communication protocol or a cellular communication protocol.

Referring still to FIG. 8, analyte monitoring system 800 further comprises a second reader device 120-2 belonging to caregiver 801-B. Second reader device 120-2 can include communication circuitry configured to wirelessly communicate data with trusted computer system 180 through network 190 via an 802.11x communication protocol or a cellular communication protocol. According to many embodiments, network 190 can be the Internet.

According to another aspect of the embodiments, sensor control device 102 includes an analyte sensor at least a portion of which is configured to be positioned in the body of patient 801-A. Sensor control device 102 can wirelessly communicate data indicative of an analyte level of patient 801-A to reader device 120-1, which in turn can wirelessly communicate the data to trusted computer system 180. According to another aspect of the embodiments, second reader device 120-2 includes analyte monitoring software configured to be operated by caregiver 801-B. Furthermore, second reader device 120-2 (which can be a smart phone) is configured to receive data indicative of an analyte level of patient 801-A, which is wirelessly communicated by trusted computer system 180 via network 190 to reader device 120-2.

In this manner, caregiver 801-B can receive timely information regarding the analyte information of patient 801-A. In some embodiments, for example, analyte monitoring software installed on the caregiver's reader device 120-2 can be configured to receive alarms associated with analyte levels of patient 801-A.

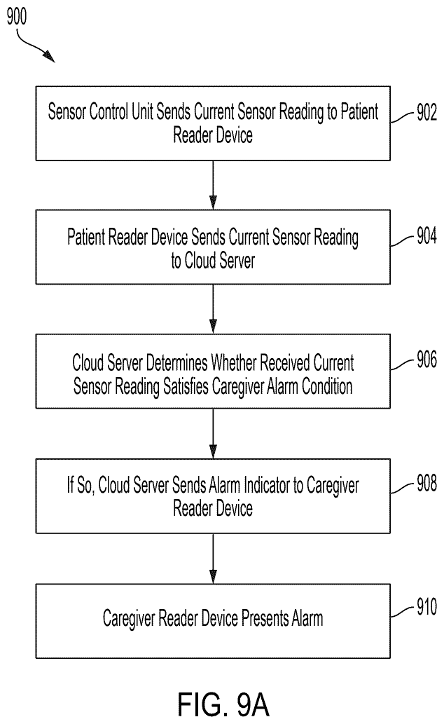

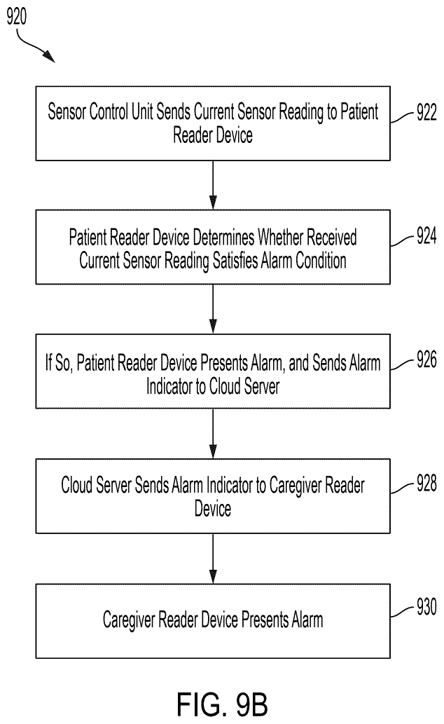

FIG. 9A is a flow diagram depicting an example embodiment of a method 900 for providing a caregiver alarm in relation to an analyte monitoring system. At Step 902, the sensor control unit worn by a patient communicates a current sensor reading to a patient's reader device. According to many of the embodiments, the patient's reader device can be a smart phone that includes communication circuitry configured to wirelessly communicate data with the sensor control unit according to a Bluetooth, Bluetooth Low Energy, or Near Field Communication protocol. Those of skill will recognize that other standard wireless communication protocols can be utilized and are fully within the scope of the present disclosure. At Step 904, the patient's reader device then transmits the current sensor reading to a cloud-based server. According to many of the embodiments, the communication circuitry of the patient's reader device can be further configured to wirelessly communicate data with the cloud-based server according to an 802.11x protocol, cellular communication protocol, or any other standard wireless communication protocol.

Referring still to FIG. 9A, at Step 906, the cloud-based server determines whether the received current sensor reading satisfies a caregiver alarm condition. According to an aspect of method 900, the caregiver alarm condition can be configurable by the caregiver and can operate independently of the patient's own alarm conditions. In some embodiments, for example, the caregiver alarm condition can be one of: a high glucose level condition, a low glucose level condition, or a signal loss condition. In other embodiments, the caregiver alarm condition can also comprise a rate-of-change condition, a predicted glucose level condition, or a failure to dismiss an alarm condition on the patient's device. At Step 908, if the received current sensor reading satisfies the caregiver alarm condition, then the cloud-based server then transmits an alarm indicator associated with the caregiver alarm condition to the caregiver's reader device. At Step 910, the caregiver's reader device presents an alarm in response to receiving the alarm indicator.

FIG. 9B is a flow diagram depicting another example embodiment of a method 920 for providing a caregiver alarm in relation to an analyte monitoring system. At Step 922, the sensor control unit worn by a patient communicates a current sensor reading to a patient's reader device. According to many of the embodiments, the patient's reader device can be a smart phone that includes communication circuitry configured to wirelessly communicate data with the sensor control unit according to a Bluetooth, Bluetooth Low Energy, or Near Field Communication protocol. Those of skill will recognize that other standard wireless communication protocols can be utilized and are fully within the scope of the present disclosure. At Step 924, the patient's reader device determines whether the received current sensor reading satisfies an alarm condition. In some embodiments, for example, the alarm condition can be one of: a high glucose condition, a low glucose condition, a signal loss condition, a rate-of-change condition, or a predicted glucose level condition, among others.

Referring still to FIG. 9B, at Step 926, if an alarm condition is satisfied, then the patient's reader device presents an alarm to the patient and, furthermore, transmits an alarm indicator associated with the determined alarm condition to the cloud-based server. At Step 928, the cloud-based server then transmits the received alarm indicator associated with the determined alarm condition to the caregiver's reader device. At Step 930, the caregiver's reader device presents an alarm based on the received alarm indicator associated with the determined alarm condition.

According to an aspect of method 920, the analyte monitoring system is configured such that the caregiver is only presented with alarms configured by the patient. In other words, according to the embodiment of method 920, the alarm indicators received by the caregiver merely “pass through” the cloud-based server without any independent evaluation performed by the either the cloud-based server or the caregiver's reader device.

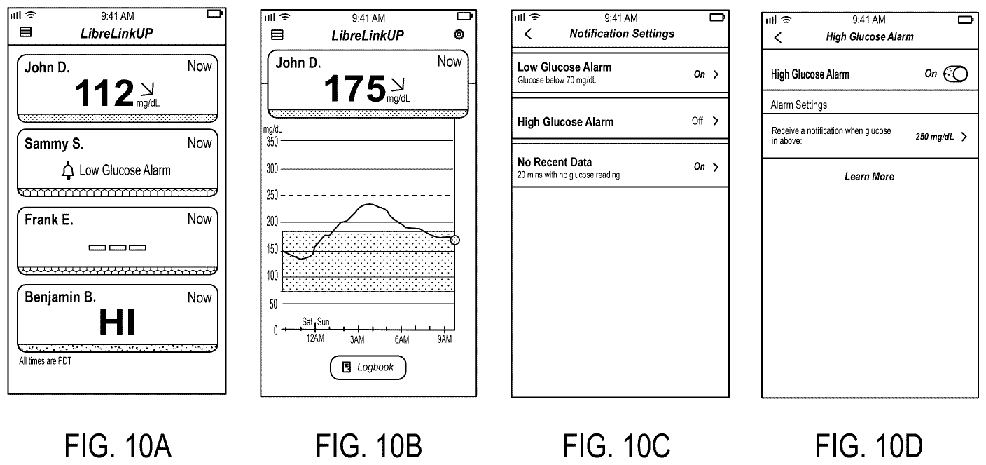

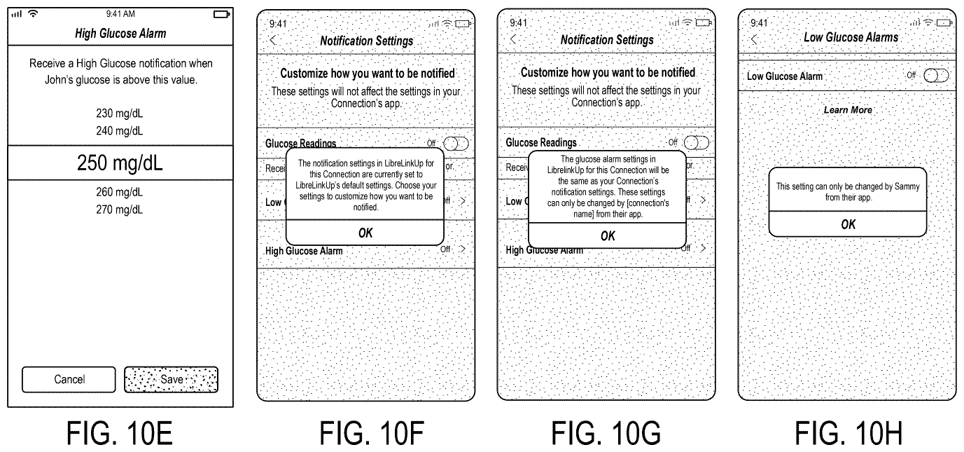

FIGS. 10A to 10H depict various GUIs and alarm interfaces of an analyte monitoring software application configured to operate on a caregiver's reader device. FIG. 10A is a GUI depicting a home interface with multiple connections, each depicted as a profile card. FIG. 10B is a GUI depicting an analyte graph for a specific connection. FIG. 10C is a GUI depicting an alarm configuration interface for toggling on/off a low glucose alarm, high glucose alarm, and a no recent data alarm. FIG. 10D is a GUI depicting another alarm configuration interface for a high glucose alarm, in which the interface indicates the current value for a high glucose alarm threshold. FIG. 10E is a GUI depicting still another alarm configuration interface for a high glucose alarm, in which the interface includes a user configurable setting for a high glucose alarm threshold. FIG. 104F is a GUI depicting an alarm configuration interface, in which the interface indicates that the alarm settings are initially set to default settings provided by the caregiver's software application. FIG. 10G is another GUI depicting an alarm configuration interface, in which the interface indicates that the alarm settings are not configurable and will be the same as the patient's alarm settings. FIG. 10H depicts an alarm configuration interface in relation to FIG. 10G, when the caregiver attempts to navigate to a specific alarm setting. In some embodiments, the alarm settings, although not configurable, can be made visible to the caregiver through the caregiver's software application.

Although many of the embodiments described herein relate to glucose monitoring, those of skill in the art will appreciate that these same embodiments can be implemented for purposes of monitoring other analytes, such as, for example, lactate and ketones.

Furthermore, those of skill in the art will appreciate that the embodiments described herein are not limited to the monitoring one analyte at a time, although each embodiment described herein is capable of doing so. For example, according to some embodiments, a single sensor control device can include within its housing, for example, an analyte sensor capable of sensing an in vivo glucose level and an in vivo lactate level in a bodily fluid of the user. Likewise, any and all of the aforementioned embodiments of processes, display windows, methods, and/or alarms can be configured for purposes of monitoring multiple analytes at once (e.g., glucose and lactate, glucose and ketone, etc.).

In summary, improved digital interfaces, graphical user interfaces, and alarms for analyte monitoring systems are provided. For example, various embodiments of interfaces for alarms for an analyte monitoring software application are described. Also, various embodiments of interface enhancements are described, including caregiver alarms, among other embodiments.

It should be noted that all features, elements, components, functions, and steps described with respect to any embodiment provided herein are intended to be freely combinable and substitutable with those from any other embodiment. If a certain feature, element, component, function, or step is described with respect to only one embodiment, then it should be understood that that feature, element, component, function, or step can be used with every other embodiment described herein unless explicitly stated otherwise. This paragraph therefore serves as antecedent basis and written support for the introduction of claims, at any time, that combine features, elements, components, functions, and steps from different embodiments, or that substitute features, elements, components, functions, and steps from one embodiment with those of another, even if the following description does not explicitly state, in a particular instance, that such combinations or substitutions are possible. It is explicitly acknowledged that express recitation of every possible combination and substitution is overly burdensome, especially given that the permissibility of each and every such combination and substitution will be readily recognized by those of ordinary skill in the art.

While the embodiments are susceptible to various modifications and alternative forms, specific examples thereof have been shown in the drawings and are herein described in detail. It should be understood, however, that these embodiments are not to be limited to the particular form disclosed, but to the contrary, these embodiments are to cover all modifications, equivalents, and alternatives falling within the spirit of the disclosure. Furthermore, any features, functions, steps, or elements of the embodiments may be recited in or added to the claims, as well as negative limitations that define the inventive scope of the claims by features, functions, steps, or elements that are not within that scope.

As referred to herein a nearby devices setting of a reader device may determine whether a software application installed on the reader device can communicate with other nearby devices, for example find, connect to, and/or determine the position of other nearby devices. More specifically, when a nearby devices setting is enabled in relation to a software application, the application may be allowed to search for devices and networks using wireless communication protocols (e.g., Wi-Fi), for example that are separate from broader location services and/or navigational systems (e.g., GPS). A nearby device may be a device which is within communication range of the reader device using the communication protocol, and/or which is connected to the same wireless network. Thus, the wireless communication circuitry may be configured to receive data indicative of the analyte level of the subject from the sensor control device only when a nearby devices setting of the reader device is enabled.

As referred to herein, the analyte monitoring software program may be an application installed on the reader device. Modifying the analyte monitoring software program may include blocking the analyte monitoring software such that no function of feature is available to a user, or restricting the availability of at least one function or feature, for example where the software is arranged to provide alarms, modifying the software to indicate that alarms are unavailable.
Exemplary embodiments are set forth in the following numbered clauses:
1. An analyte monitoring system, comprising:
a sensor control device comprising an analyte sensor, wherein a portion of the analyte sensor is configured to be positioned through a skin surface of a subject and in fluid contact with a bodily fluid of a subject, and wherein the portion of the analyte sensor is further configured to sense an analyte level in the bodily fluid; and
a reader device, comprising:
   wireless communication circuitry configured to receive data indicative of the analyte level of the subject from the sensor control device; and
   one or more processors coupled to a memory, the memory storing an analyte monitoring software program that, when executed by the one or more processors, causes the one or more processors to:
      monitor a nearby devices setting,
      determine whether the nearby devices setting is enabled, and
      in response to a determination that the nearby devices setting is not enabled, modify the analyte monitoring software program.
2. The analyte monitoring system of clause 1, wherein the analyte monitoring software program, when executed by the one or more processors, causes the one or more processors to monitor the nearby devices setting periodically while the analyte monitoring software program is running.
3. The analyte monitoring system of clause 1 or 2, wherein the analyte monitoring software program, when executed by the one or more processors, causes the one or more processors to monitor the nearby devices setting when the analyte monitoring software program is starting.
4. The analyte monitoring system of clause 1, 2 or 3, wherein the analyte monitoring software program, when executed by the one or more processors, causes the one or more processors to monitor the nearby devices setting in response to one or more predetermined events.
5. The analyte monitoring system of clause 4, wherein the one or more predetermined events comprise viewing an alarms settings graphical user interface (GUI) of the analyte monitoring software program.
6. The analyte monitoring system of clause 4 or 5, wherein the one or more predetermined events comprise navigating from a first graphical user interface (GUI) of the analyte monitoring software program to a second GUI of the analyte monitoring software program.
7. The analyte monitoring system of any preceding clause, wherein the analyte monitoring software program, when executed by the one or more processors, causes the one or more processors to modify the analyte monitoring software program, in response to a determination that the nearby devices setting is not enabled, by blocking further operation of the analyte monitoring software program until the nearby devices setting is enabled.
8. The analyte monitoring system of clause 7, wherein blocking further operation of the analyte monitoring software program includes restricting availability of at least one function or feature of the analyte monitoring software program.
9. The analyte monitoring system of any preceding clause, wherein the analyte monitoring software program, when executed by the one or more processors, causes the one or more processors to modify the analyte monitoring software program, in response to a determination that the nearby devices setting is not enabled, by displaying an indication that one or more alarms are unavailable.
10. The analyte monitoring system of clause 9, wherein the indication that one or more alarms are unavailable comprises an in-app banner notification.
11. The analyte monitoring system of clause 10, wherein the in-app banner notification is configured to navigate to an operating system settings interface.
12. The analyte monitoring system of any preceding clause, wherein the nearby device settings is configured to determine whether the analyte monitoring software program can communicate with one or more nearby devices.
13. The analyte monitoring system of clause 12, wherein the analyte monitoring software program communicating with one or more nearby devices includes finding one or more other nearby devices, connecting to one or more other nearby devices, or determining a position of one or more other nearby devices.
14 The analyte monitoring system of clause 12 or 13, wherein the one or more nearby devices includes one or more devices within a communication range of the reader device, wherein the one or more nearby devices includes one or more devices that use a communication protocol that is separate from a broader location services or a navigational systems.
15. The analyte monitoring system of clause 14, wherein the communication protocol is a wireless communication protocol, wherein the wireless communication protocol is a Wi-Fi protocol.
16. The analyte monitoring system of clause 14, wherein the broader location services or the navigational systems includes a GPS system.
17. The analyte monitoring system of clauses 12, 13 or 14, wherein the one or more nearby devices includes one or more devices within a communication range of the reader device, wherein the one or more nearby devices includes one or more devices more devices that are connected to a same wireless network as the reader device.
18. The analyte monitoring system of any preceding clause, wherein, in response to a determination that the nearby devices settings is enabled, the analyte monitoring software program, when executed by the one or more processors, causes the one or more processors to search for one or more devices and one or more networks using one or more wireless communication protocols that are separate from a broader location services or a navigational systems.
19. The analyte monitoring system of any preceding clause, wherein the wireless communication circuity is further configured to receive data indicative of the analyte level of the subject from the sensor control device only in response to a determination that the nearby devices settings is enabled.
20. An analyte monitoring system, comprising:
a sensor control device comprising an analyte sensor, wherein a portion of the analyte sensor is configured to be positioned through a skin surface of a subject and in fluid contact with a bodily fluid of a subject, and wherein the portion of the analyte sensor is further configured to sense an analyte level in the bodily fluid; and
a reader device, comprising:
   wireless communication circuitry configured to receive data indicative of the analyte level of the subject from the sensor control device; and
   one or more processors coupled to a memory, the memory storing an analyte monitoring software program that, when executed by the one or more processors, causes the one or more processors to:
      monitor a device alarms settings for a silent mode feature,
   determine whether the silent mode feature is enabled in the device alarms settings,
   in response to a determination that the silent mode feature is enabled, disable alarms from outputting an audible noise on the analyte monitoring software program,
   determine an expiration time for the silent mode feature, wherein the expiration time is a predetermined time at which the silent mode feature is disabled, and
   output a notification related to the silent mode feature when at least half a duration of the expiration time has elapsed.
21. The analyte monitoring system of clause 20, wherein the notification is outputted silently to the subject.
22. The analyte monitoring system of clause 20 or 21, wherein the notification is configured to indicate to the subject the duration of time remaining until the expiration time is reached.
23. The analyte monitoring system of clause 20, 21 or 22, wherein the notification is configured to indicate to the subject that the silent mode feature is enabled.
24. The analyte monitoring system of any of clauses 20 to 23, wherein the reader device further comprises a touchscreen, and wherein the analyte monitoring software application, when executed by the one or more processors, further causes the one or more processors to:
   in response to a predetermined input by the subject on the notification,
   output a modal, wherein the modal provides the subject with information related to the device alarm settings for the silent mode feature.
25. The analyte monitoring system of clause 24, wherein the modal comprises a list indicating which alarms are disabled from outputting the audible noise.
26. The analyte monitoring system of clause 24 or 25, wherein the modal comprises a countdown time indicating the duration of time remaining until the expiration time.
27. The analyte monitoring system of clause 24, 25 or 26, wherein the modal comprises a textual indicator to indicate the expiration time of the silent mode feature.
28. The analyte monitoring system of any of clauses 24 to 27, wherein the modal comprises a first button, wherein a first predetermined increment of time is added to the expiration time upon the subject selecting the first button.
29. The analyte monitoring system of clause 28, wherein the first predetermined increment of time is five minutes.
30. The analyte monitoring system of any of clauses 20 to 29, wherein the modal comprises a second button, wherein a second predetermined increment of time is reduced from the expiration time upon the subject selecting the second button.
31. The analyte monitoring system of clause 30, wherein the second predetermined increment of time is five minutes.
32. The analyte monitoring system of any of clauses 20 to 31, wherein the modal comprises a third button, wherein the silent mode feature is configured to be disabled upon the subject selecting the third button.
33. The analyte monitoring system of any of clauses 20 to 32, wherein the expiration time is a minimum time limit of five minutes.
34. The analyte monitoring system of any of clauses 20 to 33, wherein the expiration time is a maximum time limit of six hours.
35. The analyte monitoring system of any of clauses 20 to 34, wherein the analyte monitoring software application, when executed by the one or more processors, further causes the one or more processors to:
prior to enabling the silent mode feature in the device alarms settings, authenticate the subject through a biometric routine.
36. The analyte monitoring system of clause 35, wherein the biometric routine includes a facial recognition routine.
37. The analyte monitoring system of any of clauses 20 to 36, wherein the analyte monitoring software application, when executed by the one or more processors, further causes the one or more processors to:
prior to enabling the silent mode feature in the device alarms settings, prompting the subject to enter a password or PIN number for authentication.
38. An analyte monitoring system, comprising:
a sensor control device comprising an analyte sensor, wherein a portion of the analyte sensor is configured to be positioned through a skin surface of a subject and in fluid contact with a bodily fluid of a subject, and wherein the portion of the analyte sensor is further configured to sense an analyte level in the bodily fluid; and
a reader device, comprising:
   wireless communication circuitry configured to receive data indicative of the analyte level of the subject from the sensor control device; and
   one or more processors coupled to a memory, the memory storing an analyte monitoring software program that, when executed by the one or more processors, causes the one or more processors to:
      monitor a device alarms settings for a temporary mode feature, wherein the temporary mode feature comprises a plurality of alarm settings options for the analyte monitoring software program,
      determine whether one of the plurality of alarm settings options is enabled in the device alarms settings,
      wherein the plurality of alarm settings options comprise:
         a silent mode option, wherein the silent mode option is configured to disable a first one or more alarms from outputting an audible noise;
         a vibrate mode option, wherein the vibration mode option is configured to enable a second one or more alarms to output vibratory alerts; and
         a full volume option, wherein the full volume option is configured to enable a third one or more alarms to output a maximum volume of the audible noise, and
      in response to a determination that one of the plurality of alarm settings options is enabled, activate the temporary mode feature corresponding to the enabled one of the plurality of alarm settings options, wherein the temporary mode feature is activated for a predetermined period of time before automatically deactivating.
39. The analyte monitoring system of clause 38, wherein the maximum volume is a volume to which a volume of the reader device is set.
40. The analyte monitoring system of clause 38 or 39, wherein the maximum volume is a preset or default volume set by the analyte monitoring software program.
41. The analyte monitoring system of any of clauses 38 to 40, wherein only one of the plurality of alarm settings options can be enabled at one time.
42. The analyte monitoring system of any of clauses 38 to 41, wherein the first one or more alarms, the second one or more alarms, and the third one or more alarms are a same one or more alarms.
43. The analyte monitoring system of any of clauses 38 to 41, wherein the first one or more alarms, the second one or more alarms, and the third one or more alarms are different.
44. The analyte monitoring system of any of clauses 38 to 43, wherein the analyte monitoring software application, when executed by the one or more processors, further causes the one or more processors to:
output a graphical user interface, wherein the graphical user interface comprises an indicator configured to indicate which one of the plurality of alarm settings options is enabled.
45. The analyte monitoring system of clause 44, wherein the indicator is a colored dot, wherein a color of the colored dot is associated with the one of the plurality of alarm settings options that is enabled.
46. The analyte monitoring system of any of clauses 38 to 45, wherein the first one or more alarms, the second one or more alarms, and the third one or more alarms are customizable by the subject.
47. The analyte monitoring system of any of clauses 38 to 46, wherein a name of each of the plurality of alarm settings option is customizable by the subject.
48. The analyte monitoring system of any of clauses 38 to 47, wherein a sound of the audible noise is customizable by the subject.
49. The analyte monitoring system of any of clauses 38 to 48, wherein a pattern of the vibratory alerts is customizable by the subject.
50. An analyte monitoring system, comprising:
a sensor control device comprising an analyte sensor, wherein a portion of the analyte sensor is configured to be positioned through a skin surface of a subject and in fluid contact with a bodily fluid of a subject, and wherein the portion of the analyte sensor is further configured to sense an analyte level in the bodily fluid; and
a reader device, comprising:
   wireless communication circuitry configured to receive data indicative of the analyte level of the subject from the sensor control device; and
   one or more processors coupled to a memory, the memory storing an analyte monitoring software program that, when executed by the one or more processors, causes the one or more processors to:
determine whether the subject is within a predetermined analyte range from the data indicative of the analyte level,
monitor the device alarms settings for a back in range feature,
determine whether the back in range feature is enabled in the device alarm settings, and
in response to a determination that the back in range feature is enabled and a determination the subject has returned to the predetermined analyte range, output a notification, wherein the notification is configured to indicate to the subject that the subject has returned to the predetermined analyte range.

1-19. (canceled)

20. An analyte monitoring system, comprising:
a sensor control device comprising an analyte sensor, wherein a portion of the analyte sensor is configured to be positioned through a skin surface of a subject and in fluid contact with a bodily fluid of a subject, and wherein the portion of the analyte sensor is further configured to sense an analyte level in the bodily fluid; and
a reader device, comprising:
wireless communication circuitry configured to receive data indicative of the analyte level of the subject from the sensor control device; and
one or more processors coupled to a memory, the memory storing an analyte monitoring software program that, when executed by the one or more processors, causes the one or more processors to:
monitor a device alarms settings for a silent mode feature,
determine whether the silent mode feature is enabled in the device alarms settings,
in response to a determination that the silent mode feature is enabled, disable alarms from outputting an audible noise on the analyte monitoring software program,
determine an expiration time for the silent mode feature, wherein the expiration time is a predetermined time at which the silent mode feature is disabled, and
output a notification related to the silent mode feature when at least half a duration of the expiration time has elapsed.

21. The analyte monitoring system of claim 20, wherein the notification is outputted silently to the subject.

22. The analyte monitoring system of claim 20, wherein the notification is configured to indicate to the subject the duration of time remaining until the expiration time is reached.

23. The analyte monitoring system of claim 20, wherein the notification is configured to indicate to the subject that the silent mode feature is enabled.

24. The analyte monitoring system of claim 20, wherein the reader device further comprises a touchscreen, and wherein the analyte monitoring software application, when executed by the one or more processors, further causes the one or more processors to:
in response to a predetermined input by the subject on the notification,
output a modal, wherein the modal provides the subject with information related to the device alarm settings for the silent mode feature.

25. The analyte monitoring system of claim 24, wherein the modal comprises a list indicating which alarms are disabled from outputting the audible noise. 26 (Original) The analyte monitoring system of claim 24, wherein the modal comprises a countdown time indicating the duration of time remaining until the expiration time.

27. The analyte monitoring system of claim 24, wherein the modal comprises a textual indicator to indicate the expiration time of the silent mode feature.

28. The analyte monitoring system of claim 24, wherein the modal comprises a first button, wherein a first predetermined increment of time is added to the expiration time upon the subject selecting the first button.

29. The analyte monitoring system of claim 28, wherein the first predetermined increment of time is five minutes.

30. The analyte monitoring system of claim 24, wherein the modal comprises a second button, wherein a second predetermined increment of time is reduced from the expiration time upon the subject selecting the second button.

31. The analyte monitoring system of claim 30, wherein the second predetermined increment of time is five minutes.

32. The analyte monitoring system of claim 24, wherein the modal comprises a third button, wherein the silent mode feature is configured to be disabled upon the subject selecting the third button.

33. The analyte monitoring system of claim 20, wherein the expiration time is a minimum time limit of five minutes.

34. The analyte monitoring system of claim 20, wherein the expiration time is a maximum time limit of six hours.

35. The analyte monitoring system of claim 20, wherein the analyte monitoring software application, when executed by the one or more processors, further causes the one or more processors to:
prior to enabling the silent mode feature in the device alarms settings, authenticate the subject through a biometric routine.

36. The analyte monitoring system of claim 35, wherein the biometric routine includes a facial recognition routine.

37. The analyte monitoring system of claim 20, wherein the analyte monitoring software application, when executed by the one or more processors, further causes the one or more processors to:
prior to enabling the silent mode feature in the device alarms settings, prompting the subject to enter a password or PIN number for authentication.

38-50. (canceled)
